(19) United States
(12) Patent Application Publication
(10) Pub. No.: US 2005/0123920 A1
(43) Pub. Date: Jun. 9, 2005

(54) Jade nucleic acids, proteins and uses thereof

(76) Inventors: Herbert Tod Cohen, Newton, MA (US); Mina I. Zhou, Brookline, MA (US); Hongmei Wang, Westborough, MA (US)

Correspondence Address: RONALD I. EISENSTEIN, 100 SUMMER STREET, NIXON PEABODY LLP, BOSTON, MA, 02110, US

(21) Appl. No.: 10/506,266

(22) PCT Filed: Mar. 6, 2003
(86) PCT No.: PCT/US03/06926

Related U.S. Application Data
(60) Provisional application No. 60/361,939, filed on Mar. 6, 2002.

(51) Int. Cl. C12Q 1/68; G01N 33/574; C07H 21/04

(52) U.S. Cl. 435/6; 435/7.23; 435/69.1; 435/320.1; 435/325; 530/350; 536/23.5

(57) ABSTRACT

The present invention provides isolated and substantially purified nucleic acids encoding human Jade-1 (for “gene” for Apoptosis and Differentiation in Epithelia) and related nucleic acids. The invention further provides Jade protein, which, when expressed in a eukaryotic cell, has an apparent molecular weight of 64 kD. We have discovered that Jade protein functions 1 apoptosis and regulation of cell cycle as well as in cellular differentiation process. Accordingly, the Jade-family of genes and their gene products provide novel diagnostic and prognostic tools for identifying and/or classifying diseases involving abnormal apoptosis and defective cell cycle.

### BACKGROUND OF THE INVENTION

Apoptosis, or programmed cell death, and its abnormalities form a significant component in various disease processes. Decreased apoptosis can lead to accumulation of malignant cells i.e. cancer, whereas increased apoptosis can be a problem in tissues after injury such as hypoxic stress, wherein destruction of cells leads to destruction of tissues and their normal function. Although a number of apoptosis regulating proteins are already known, it would be desirable to identify additional molecules involved in this fundamental regulatory process to enable diagnosis and development of treatment methods for diseases treatment of which could benefit from regulation of apoptosis.

In addition to apoptosis, defects in the cell cycle resulting in abnormal cell proliferation and differentiation are another major cause of human diseases.

For example, cancer is a major clinical problem in present society and both problems in apoptosis and cell cycle regulation have been identified in numerous different cancers affecting humans. Problems not only include treatment but also diagnosis of different types of cancers. For example, there are a number of different cancers that can be successfully treated if the cancer is discovered early enough. Moreover, the pharmacogenetic and pharmacogenomic methods have increasingly indicated that different defects causing cancer are responsive to different kinds of treatment regimes. Thus, it would be desirable to have additional markers that can be used for diagnosis and/or prognosis of cancers and that could provide guidance in selecting a treatment regime effective to treat a particular type of cancer.

Additionally, while developments in chemotherapy have been improving survival rates and remedial rates of patients having neoplastic diseases, strong side effects of carcinostatic agents can cause serious damage on normal cells resulting in severe side effects of carcinostatic agents. Therefore there remains demand for agents which have selectivity for cancer cells or which are capable of controlling proliferation of oncocytes and present less severe side effects.

In conventional chemotherapy for cancer, administration dosage of agents is kept as low as possible to prevent harmful side effects. Thus, attempts have been made to seek potentiation by combining a plurality of carcinostatic agents with different mechanisms of actions, or to improve carcinostatic effect by combining a carcinostatic agent with other substances. In the latter case, a carcinostatic agent is usually combined with an immune activator to combine the direct effect on oncocytes with antineoplastic effect obtained through activation of immunocompetence of an organism. Further, in some cases, radiotherapy or surgical treatment is performed in addition to these methods to improve the effect of the treatment. Unfortunately there still remain several types of cancers which respond poorly to currently available therapies.

For example, treatment of renal cancer with currently existing therapeutic interventions has been poor. About 30,000 renal cancer cases and 12,000 deaths are reported annually in the US, making it the 8^{th} leading cancer. Roughly 80% of cases are due to clear-cell renal cancer, which often presents with a metastatic disease. Advanced renal cancer responds poorly to therapy. Only 20% of patients show improvement with interleukin-2, although early trials with tumor vaccines are encouraging (10). Assays to identify new compounds that interact and modulate certain cancers, particularly renal cancers would be desired.

In addition to cancers, cell cycle defects are a cause also of other types of diseases. For example, in the United States, about 600,000 people have polycystic kidney disease (PKD), a genetic disorder characterized by the growth of numerous cysts in the kidneys. PKD is the fourth leading cause of kidney failure. PKD can cause cysts in the liver and problems in other organs, such as the heart and blood vessels in the brain. These complications help doctors distinguish PKD from the usually harmless “simple” cysts that often form in the kidneys in later years of life. When PKD causes kidneys to fail, which usually happens only after many years, the patient requires dialysis or kidney transplantation. About one-half of people with the major type of PKD progress to kidney failure, i.e., end-stage renal disease (ESRD). Currently, there exists no treatments for PKD except for a kidney transplant.

Therefore, further identification of proteins and methods for modulating apoptosis and cell cycle would provide more accurate means of diagnosis, prognosis and treatment of diseases preferably in areas where such cellular regulatory pathways are affected, including cancer and renal cysts.

### SUMMARY OF THE INVENTION

We have now isolated nucleic acids that encode a novel family of human tumor suppressors Jade-1 and Jade-1 fragments and derivatives capable of hybridizing to such Jade-1-encoding nucleic acids. We have now isolated a cDNA (SEQ ID NO:4) encoding human Jade-1 protein (SEQ ID NO: 5) and have expressed the cDNA in eukaryotic cells, resulting in recombinant Jade-1 protein having an apparent molecular weight of about 64 kDa.

The invention further provides isolated and substantially pure recombinant human Jade-1 and Jade-1 fragments and derivatives thereof. These fragments preferably contain at least about 85% homology to SEQ ID NO:4, more preferably at least about 90% homology, still more preferably at least about 95% homology.

In one preferred embodiment, the Jade nucleic acid sequences of the present invention encode at least 21, preferably at least 25, 30, 50, and most preferably at lest 100, 200 consecutive amino acids of the SEQ ID NO: 5,

In another preferred embodiment, the nucleic acid fragments of the present invention exclude nucleic acids encoding amino acids 75-83; 182-201; 217-224; 225-245; 276-288; 332-340; 343-350; 354-362; 433-439; 441-446; and 447-482 of the clone KIAA0215 with an Entrez nucleotide accession number D86969. In another preferred embodiment, the isolated an purified nucleic acids exclude fragments 107-121; 135-151; 249-264 of the KIAA0239 gene with accession number gi27529704.

In another preferred embodiment, the invention provides a nucleic acid encoding a protein comprising 5 or more consecutive amino acids 1-142 and/or 503-509 as shown in SEQ ID NO: 5, and at least one functional domain of SEQ ID NO: 5.

In yet one preferred embodiment the invention provides a nucleic acid sequence which does not encode the amino acids of 1-362 of the clone KIAA1807 with an Entrez nucleotide accession number AB058710.

The invention also provides novel assays for identifying compounds useful in the diagnosis or treatment of disorders related to the apoptotic activity associated with Jade-1 expressing cells. Preferred compounds identified through assays of the invention can modulate, preferably mimic or enhance, Jade-1 activity and thus can be used to induce the apoptotic activity associated with Jade-1 expressing cells.

In another preferred embodiment, the invention provides a method of decreasing apoptosis by decreasing Jade activity in tissues with normal cells affected by a hypoxic event. In the preferred embodiment, the Jade activity is decreased using compounds selected from the group consisting of antibodies raised against Jade protein, antisense oligonucleotides against Jade transcript, interference RNAs (iRNA) directed against Jade transcript, and small organic and inorganic molecules.

In yet another embodiment, the invention provides methods of increasing Jade expression in stem cells or neural stem cells to promote neurogenesis.

As used herein, “Jade-1 activity” refers to at least one particular activity displayed by a construct having the wild type nucleic acid sequence when its expression results in a protein that has the native conformation encoding Jade.

The invention further provides a variety of assays to identify agents capable of regulating cell proliferation and/or differentiation and apoptosis by increasing or decreasing Jade activity. Agents identified through assays of the invention will have potential for use in a number of therapeutic applications, especially to modulate, particularly enhance or mimic, expression or activity of Jade-1 in particular cells, preferably cancer cells. Alternatively, binding assays can be used to identify agents that bind to Jade-1 and inhibit its interaction with, and thus may be used to inhibit Jade-1 induced apoptosis, for example Jade/VHL interaction. Moreover, assays for identifying agents inhibiting or enhancing Jade-induced chromosome acetylation, and Jade-induced apoptosis are provided.

Specific diseases and disorders that could be treated by administration of pharmacological agents that enhance Jade-1 expression or mimic Jade-1 function identified through assays of the invention include those associated with cancer such as renal cancer, breast cancer, cervical, uterine and prostate cancers, liver cancer, colon cancer, leukemias and lymphomas; VHL disease; cystic renal disease, such as ADPKD; and in other cancers and diseases in which pathogenesis apoptosis and/or cell cycle defects affected by Jade or its homologues play a role.

The invention further provides a method of decreasing Jade activity in normal cells undergoing undesired apoptosis. For example after a catastrophic tissue injury caused by renal failure or hypoxic event apoptosis frequently occurs. However, in those situations where cell survival is desirable such other similar situations include ischemia, for example in the myocardium following myocardial infarction, in the brain following cerebrovascular events, or in chronic injury states, such as in fibrosis.

The invention further provides a method of preventing cell death and increasing differentiation of neurons by increasing the amount of Jade in the target tissue. Therefore, the invention provides a method of treating neurogenerative diseases. For example, control of an ER stress response by Jade proteins is particularly important in Alzheimer's disease, where the neurons have been,shown to die via apoptotic pathway, or in cardiovascular disease in response to homocysteine.

The invention further provides a method of prevention and treatment of cancer by providing Jade gene or Jade protein members of the Jade family, or functional fragments or homologues thereof into a subject in need thereof wherein increase of Jade expression to a level at least close to normal levels in such tissues is indicative of effective treatment.

The invention further provides a method of treating or preventing cancer, preferably renal cancer, VHL disease, and renal cystic disease, including ADPKD, using proteasome inhibitors capable of increasing Jade protein expression.

In one embodiment, the invention provides antibodies against Jade members of the Jade family, such at proteins or antigenic epitopes thereof. These antibodies are useful in diagnosis and/or prognosis of cancers and renal cystic diseases using the Jade family, increasing therapies such as treatment with proteasome inhibitors. The antibodies are further useful in methods of screening for agents capable of increasing or decreasing Jade amount in cells or tissues in vitro or in vivo.

Other aspects of the invention are disclosed infra.

### BRIEF DESCRIPTION OF THE FIGURES

FIG. 1 shows the Jade-1 protein amino acids (aas) 1-509 (SEQ ID NO.:5). PEST domain (aas 5-28) by PESTfind is underlined. Consensus PHDs (203-253, and 312-371 aas) by NCBI-BLAST and BLOCK search are bold and underlined. Candidate Hox domain (aas 400-462) by NCBI-BLAST is bold.

FIG. 2 shows the complete coding sequence of the human Jade-1 consisting essentially of nucleotides 1-1533 (SEQ ID NO.: 4). Initiating ATG (bold) is at position 1 and 2 stop codons (bold) are at the 3′ end. A complete human Jade-1 coding sequence clone was identified from the human kidney cDNA library. Human EST sequences were compared to identify the most 5′ nucleotides to help denote the intiating ATG and also the complete cDNA clone.

FIGS. 3A-3B illustrates a complete cDNA clone of the human Jade-1. Nucleotides 1-3585 (SEQ ID NO: 7) including polyA+, initiating ATG and 2 stop codons are shown in bold.

FIG. 4 shows a schematic representation of Jade-1 protein showing PEST and PHD regions and candidate homeodomain.

FIGS. 5A-5B shows coimmunoprecipitation of transfected Jade-1 and VHL. A. Immunoprecipitation of HA-VHL allows detection of cotransfected library clones FL-Jade-1 and FL-10, although the interaction is stronger with FL-Jade-1. B. Immunoprecipitation of library clones permits detection of cotransfected VHL. Again, the interaction between Jade-1 and VHL is strong.

FIG. 6 shows a Western blot of multiple mouse tissues using affinity-purified anti-peptide Jade-1 antiserum. Prominent expression of ˜60 kDa Jade-1 protein is detectable in kidney and less is in liver. The pancreas sample may reflect degradation.

FIG. 7 shows a human multi-tissue Jade-1 Northern blot showing 2 alternative splices. Predominant expression of 3.6 kb Jade-1 is in kidney, pancreas and skeletal muscle.

FIGS. 8A-8C shows coimmunoprecipitation of endogenous Jade-1 with transfected VHL. A. An affinity purified antipeptide antiserum is able to specifically immunoprecipitate Jade-1 protein and can be blocked using the immunizing peptide. The size of the immunoprecipitable endogenous Jade-1 from 293 cells is identical to immunoprecipitable transfected untagged Jade-1 (not shown), confirming the endogenous protein's identity. B. VHL antiserum can coimmunoprecipitate endogenous Jade-1 in VHL-transfected 293 cells. C. Jade-1 antiserum, but not preimmune serum, can coimmunoprecipitate transfected VHL.

FIGS. 9A-9B show that Jade-1 is upregulated by VHL and differentiation. FIG. 7A shows that Jade-1 protein is upregulated by VHL in 3 of 3 renal cancer cell lines in comparison with untransfected or empty vector cells. FIG. 7B shows that Jade-1 protein expression is high in differentiated proximal tubule cells. On the left, endogenous Jade-1 protein is upregulated with differentiation of SV40 T antigen-transformed mouse proximal tubule cells (dark arrow), and a smaller, perhaps related band is downregulated (lower arrow). On the right, fresh kidney cortex has prominent Jade-1 expression that largely disappears with primary culture, whereas the lower band becomes more prominent. The lower band is strongly downregulated by VHL in the renal cancer lines, but is not immunoprecipitable. This protein does not appear to be a Jade-1 cleavage product and likely represents a related protein.

FIG. 10 shows complete colocalization of transiently transfected Jade-1 and VHL in 293 cells. There is no signal bleed-through to either channel.

FIGS. 11A and 11B show localization of Jade-1. FIG. 9A demonstates transfected, tagged Jade-1 localization to cytoplasm and nuclear speckles using a mono-specific anti-tag antibody. FIG. 9B shows that prominent nuclear speckles are also observed using the affinity purified anti-Jade-1 serum in renal cancer cells, as well as diffuse cytoplasmic staining and cytoplasmic speckles.

FIG. 12 shows the nuclear speckles suggest Jade-1 may play a role in regulating gene expression, as do other PHD proteins. Colocalization of endogenous Jade-1 and stably transfected VHL.

FIG. 13 shows diffuse nuclear VHL and Jade-1 localization in a cell, as well as colocalization to cytoplasmic speckles in the same cell. VHL in cytoplasmic speckles colocalizes with endoplasmic reticulum markers (37).

FIG. 14 shows that transiently transfected Jade-1 causes apoptosis that can be blocked by VHL, n=5.

FIG. 15 shows that antisense (AS) Jade-1 can protect against apoptosis due to serum depletion, a context in which VHL is protective, n=3. These findings are correlated by Western blot in FIG. 14.

FIG. 16 shows a Western analysis showing effects of antisense Jade-1 on serum depletion-induced apoptosis. Top: Endogenous Jade-1 protein reduction with antisense (AS) Jade-1 expression vector. Serum depletion (SD) alone decreases Jade-1. Middle: Antisense Jade-1 reduces PARP cleavage in response to serum depletion. Bottom: Antisense Jade-1 partially restores endogenous Bcl2 levels in response to serum depletion.

FIG. 17 shows that VHL increases abundance of cotransfected Jade-1. Although not substantially different 1 day post-transfection, Jade-1 expression is increased by VHL 3 days post-transfection, which is particularly marked in fibrosarcoma cells.

FIG. 18 demonstrates that cotransfected VHL (V) increases the Jade-1 protein half-life in 293 cells. One day following transient transfection, cells were metabolically labeled for 1 hr, then chased with unlabeled methionine and cysteine for times shown. Jade-1 was immunoprecipitated from cell lysates. Jade-1 turnover is rapid. VHL readily associates with Jade-1 and prolongs its expression, increasing Jade-1 half-life by ˜2-fold.

FIG. 19 shows low-level stable VHL transfection may protect endogenous Jade-1 protein from proteasome-mediated degradation. Growth to late confluence on plastic or type-I collagen (Col I) substantially increases Jade-1 expression in VHL plus, but not in VHL minus cells. Proteasome and calpain I inhibition (PI) increase endogenous Jade-1 expression particularly in VHL minus cells at early confluence (EC). Thus, VHL likely increases Jade-1 abundance in part by inhibiting a proteasome and/or calpain pathways.

FIG. 20 shows that increased Jade-1 protein expression at high-density on collagen correlates with VHL status and mutations that do not cause renal cancer. Comparison of endogenous Jade-1 expression (arrow) by Western blot in different 786-O renal cancer cell lines grown to early confluence on plastic versus high density on collagen. Unlike renal cancer causing VHL mutations, wild-type (wt) VHL and mutations G93D and Y98H that do not cause renal cancer can upregulate Jade-1. These results support a relationship between active VHL tumor suppressor function and Jade-1 upregulation at high cell density.

FIG. 21 shows that stably-transfected VHL increases endogenous Jade-1 protein half-life in 786-O renal cancer cells. Cells were metabolically labeled for 2 hrs with ³⁵S-methionine and -cysteine, which was chased for times shown. Endogenous Jade-1 was immunoprecipitated using Jade-1 antiserum. Immunoprecipitates were run on an SDS-PAGE gel, which was dried and subject to autoradiography, shown below. Densitometry of the highly-specific Jade-1 band was performed using NIHImage 1.62 and is shown above. VHL doubles the half-life of endogenous Jade-1 protein. The increased Jade-1 level at time O in VHL+ versus VHL− cells reflects the more rapid degradation of Jade-1 without VHL through the labeling period, a hypothesis confirmed using 10 minute labeling in transiently transfected cells.

FIG. 22 shows that VHL mutations that do not cause renal cancer stabilize Jade-1 protein. On the left, as described in FIG. 15, Jade-1 transiently transfected with different VHL mutations is metabolically labeled and stabilized by wtVHL, VHL Y98H, and Y112H, which do not cause renal cancer. Considerably less stabilization is observed with VHL L118P, R167W, and the 96-122 domain deletion. The latter deletion removes the hydrophobic core of the VHL beta domain. Labeling was performed for 1 hr, followed by chase periods of 0 and 1 hrs, as denoted. Densitometric data shown as the mean of 2 experiments is shown on the right. As shown, in the presence of cotransfected wild-type VHL, there is six times more metabolically labeled Jade-1 present following the 1 hour chase period than is present with vector cotransfection. There is substantially more labeled Jade-1 at 1 hour with cotransfection of the Y98H and Y112H missense mutations than with the renal cancer-causing L118P and R167W missense mutations. Data are nicely reproducible and will be compared by ANOVA with higher experimental number.

FIG. 23 shows that wild-type VHL and mutations that do not cause renal cancer stabilize Jade-1 well, whereas renal cancer-causing VHL mutations do not. Pulse-chase metabolic labeling experiments were performed in transiently transfected 293 cells. At 0 and 1 hour chase times, Jade-1 immunoprecipitations were performed. Radiolabeled Jade-1 bands were quantitated by autoradiography and densitometry (NIHImage 1.62). Protein half-life was calculated by linear regression. Mean transfected Jade-1 protein half-lives are shown from 3 experiments ±SD in response to different VHL missense mutations shown. Non-renal cancer-causing VHL mutations Y98H, Y112H (and wt VHL) stabilize Jade-1 well, whereas renal cancer-causing mutations L118P, R167W and empty vector do not. The del96 VHL lacks amino acids 96-122, thereby missing the hydrophobic core of the VHL beta domain.

FIG. 24 shows increased Jade-1 protein expression by hypoxia. Jade-1 Western analysis was performed following 8 and 15 hours of hypoxia in 293 cells. By densitometry, there is a 3-10-fold increase in Jade-1 expression in response to hypoxia. This experiment has been performed 3 times with similar results. Thus, VHL-interacting Jade-1 protein is induced by hypoxia. Not shown, Ponceau S staining of the membrane indicates that protein loading was equal in this experiment.

FIGS. 25A and 25B show that proteasome inhibition markedly increases Jade-1 protein levels in cell from different types of cancer. Renal cancer cell lines A704, CaKi-1, and RCC4, and HT1080 fibrosarcoma cells, and colon cancer line SW620 were treated with specific proteasome inhibitors (PI) MG132 (FIG. 25A) or lactacystin beta lactone (FIG. 25B), specific calpain inhibitor (CI) PD150606 or both for 16 hours. MG132 and lactacystin are proteasome inhibitors of different classes. Both primarily inhibit proteasome chymotryptic-like protease activity, as does MLN341, and have different secondary specificities. Thus, Jade-1 degradation is likely to be due largely to proteasomal chymotryptic activity.

### DETAILED DESCRIPTION OF THE INVENTION

We have now isolated a cDNA encoding human Jade-1 (for “gene” for Apoptosis and Differentiation in Epithelia). This cDNA is represented by SEQ ID NO:4 and encodes a protein (SEQ ID NO: 5) that when expressed in a eukaryotic cell such as COS cells has an apparent molecular weight of 64 kD as determined by polyacrylamide gel electrophoresis. We have discovered that this novel protein functions in apoptosis and regulation of cell cycle as well as in cellular differentiation process. Accordingly, the Jade-family of genes and their gene products provide novel diagnostic and prognostic tools for identifying and/or classifying diseases involving abnormal apoptosis and defective cell cycle.

In one embodiment the invention provides an isolated and purified Jade-1 protein (SEQ ID NO: 5) and the nucleic acid encoding such protein (SEQ ID NO: 4 and SEQ ID NO: 7). Another aspect of the invention relates to isolated DNA segments which hybridize under stringent conditions to a DNA fragment having the nucleotide sequence set forth in SEQ ID NO: 4, or a unique fragment thereof and codes for a member of a mammalian Jade gene family. Stringent hybridization conditions are well known to the skilled artisan and are exemplified, infra. Peptides encoded by such fragments are also disclosed. Preferably, the peptide and the fragments have a wild type activity. The fragments preferably do not include the nucleic acids encoding peptide segments consisting of just amino acids 75-83; 182-201; 217-224; 225-245; 276-288; 332-340; 343-350; 354-362; 433-439; 441-446; and 447-482 of the KIAA0215 gene with a nucleotide accession number D86969 or 107-121; 135-151; 249-264 of the KIAA0239 gene with accession number gi27529704 (Entrez database at http://www.ncbi.nlm.nih.gov/entrez/query.fcgi?CMD=&DB=nucleotide). In one embodiment, the fragments do no include nucleic acids encoding 1-362 of the clone KIAA1807 with a nucleotide accession number AB058710.

In one preferred embodiment, the Jade nucleic acid sequences of the present invention encode at least 21, preferably at least 25, 30, 50, and most preferably at least 100, 200 consecutive amino acids of the SEQ ID NO: 5. In another preferred embodiment, the nucleic acid sequence of the present invention encode a sequence comprising at least 5 consecutive amino acids selected from amino acids 1-140 and 502-509 of SEQ ID NO: 5 and at least one functional domain encoded by the SEQ ID NO: 5.

We have discovered that Jade-1 and its homologues have a significant functional role in regulating apoptosis. We discovered that several proteasome inhibitors, including compounds with MLN341-like activity, significantly induced expression of Jade-1. Furthermore, when we added antisense oligonucleotide against Jade-1 to cells, cell death in these cells dropped significantly (at least about 80% compared to a control without the antisense or treated with a nonsense oligonucleotide).

Proteasomes play an important role in apoptosis and proteasome activity is necessary for cell division and growth. It is known that intracellular proteolysis occurs via two pathways: a lysosomal pathway and a non-lysosomal ATP-dependent pathway. The latter, which is known to degrade most cell proteins including regulatory proteins involves first covalent linking of proteins to multiple molecules of the polypeptide ubiquitin. This modification has the effect of marking the protein for rapid degradation by the proteasome. Proteasome 26S (200 kD) is a complex which, in mammalian cells, contains a 20S (673 kD) proteasome as the key proteolytic component, and a 19S complex containing several ATPases and a binding site for ubiquitin chains. The role of this 19S particle, which “caps” each extremity of the 20S proteasome is to unfold the protein substrates to inject them into the 20S proteasome and to stimulate the proteolytic activity. Proteasomes include both cytosolic and nuclear proteinases and have three major catalytic acitivites: a chymotrypsin-like activity, a trypsin-like activity and a post-glutamyl peptide hydrolyzing activity. Inhibition of proteasomes, particularly of their chymotrypsin-like activity, has been shown to induce apoptosis by yet an unidentified mechanism.

Without wishing to be bound by a theory, we believe that inhibition of the proteasome activity induces expression of Jade family members, and that Jade proteins directly regulate apoptosis. Therefore, induction and/or molecular mimicking of members of the Jade family, Jade and its homologues will directly result in increased apoptosis thereby providing a system to more specifically induce apoptosis of cancer cells.

Conversely, inhibition or down-regulation of members of the Jade family preferably Jade and its homologues is useful to decrease apoptosis in conditions wherein apoptosis causes undesired effects such as after hypoxia induced tissue injury or ischemia.

We have also discovered that Jade is regulated by the wildtype polycystin-1 protein. Currently, about 600,000 individuals in the U.S. are affected with autosomal dominant polycystic kidney disease (ADPKD). About 80% of the ADPKD carry a mutation in polycystin-1. We have discovered that mutant polycystin-1 is unable to regulate Jade. Therefore, proteasome inhibitors provide a means to increase members of the Jade family, preferably Jade-1 in patients with ADPKD to induce apoptosis and provide a novel treatment of ADPKD.

We have further discovered, that when Jade expression alteres several genes involved in neurogenesis. We have also discovered that Jade expression can also induce epithelial cell and muscle cell differentiation. Again, without wishing to be bound by a theory, we believe that the cell differentiation function in Jade is regulated via the PHD functional domains in Jade.

Therefore, in one preferred embodiment, the invention provides a method of increasing neurogenesis by inducing stem cells, including embryonal stem cells and neuronal stem cells, to differentiate into neurons either in vitro or in vivo by administering a Jade family protein of its neurogenesis inducing fragment or homologue to the cells, preferably the protein is Jade protein. One can also use a vector encoding such protein or a neurogenesis inducing fragment thereof.

We have further discovered that Jade expression is strongly induced by hypoxia, thereby inducing apoptosis in the hypoxic tissue. Therefore, in one embodiment, the invention provides a method of treating ischemia by either administering Jade inhibitors, such as antibodies, antisense molecules, interference RNA (iRNA), small organic or inorganic molecules which have been identified to inhibit Jade protein activity, or agents which promote destruction of Jade family of proteins to an individual suffering from an ischemic event to reduce the amount of cell death thereby reducing the amount of damage to the ischemic tissue.

We have also shown that Jade affects the cell cycle. While adding Jade protein to tumor cells increases apoptosis it also seems to result in normalizing the cell cycle. Therefore, changes in Jade expression after injury, would result in beneficial cell proliferation and differentiation by helping the cells to pass through the cell cycle. Consequently, recovering from a catastrophic tissue injury, such as an acute renal failure, where cell proliferation and differentiation would be important to repopulate the tubule basement membrane that has been destroyed, it would be advantageous to change level of Jade to help the normal cells move through cell cycle. Therefore, in one embodiment, the invention provides a method of inducing cell differentiation by administering a Jade family member or a cell differentiation promoting fragment or homologue thereof into the cell, preferably Jade or a cell differentiation promoting fragment thereof.

Further, one embodiment of the invention provides a method of inducing apoptosis in a cell affected with a cell cycle abnormality in vivo or in vitro by administering to the cell a Jade activity increasing amount of proteasome inhibitors. The cell cycle abnormalities contemplated by the present invention include, but are not limited to cancer, particularly renal, breast, and colon cancer, as well as ADPKD. A cell cycle defect susceptible to treatment by the method of the present invention can be easily identified by measuring Jade levels in the affected cells. Preferably, Jade expression is measured using immunohistochemistry, using methods well known to one skilled in the art with antibodies against Jade as discussed elsewhere in the specification. Alternatively, Jade levels can be determined using quantification of Jade mRNA. Decreased level of Jade expression as compared to normal cells of the same tissue is indicative of a cell cycle abnormality susceptible for treatment by increasing Jade protein amount in the cell.

Therefore, in one preferred embodiment, the invention provides a method of following up the efficacy and/or determining an effective dose of proteasome inhibitors in treatment of cancers and ADPKD by monitoring Jade family of protein expression levels, wherein the increase of Jade protein in the tissues of the patients compared to expression before treatment indicates a clinically sufficient amount of proteasome inhibitor to induce apoptosis preferably the family member is Jade. Jade expression is preferably determined from a biological sample taken from an individual being treated with proteasome inhibitors using immunohistochemical methods known to one skilled in the art and using Jade specific antibodies.

In one preferred embodiment, the invention also provides a method of detecting whether a disease or disorder is susceptible to treatment with proteasome inhibitors capable of increasing Jade family expression level by determining the Jade expression level in the tissue sample, for example a tumor biopsy, for example a renal tumor. Decrease in Jade expression compared to a normal tissue of the same type is indicative of a condition which can be treated by proteasome inhibitors capable of increasing Jade expression level. For example, in case of renal tumors or renal cysts, renal cells are used as a control. Preferably, the family member is Jade.

As discussed above, the invention provides methods to modulate, either to enhance or decrease expression or mimic or inhibit the activity of Jade family members such as Jade-1 in particular cells. For example, to enhance Jade-1 expression, one can use a Jade-1 encoding nucleic acid segment operably linked to a promoter to selectively direct it to desired cells or small organic or inorganic molecules identified using the methods provided by this invention. As another example, one can administer a Jade-1 protein or fragment or derivative to modulate Jade-1 activity. Administration of antibodies to Jade or immunogenic fragments thereof are the preferred method of inhibiting Jade activity. Alternatively, iRNA, antisense oligonucleotides or agents capable of promoting Jade destruction may be used.

Jade protein activity can also be modulated using proteasome inhibitors. “Proteasome inhibitor” shall mean any substance which directly or indirectly inhibits the proteasome or the activity thereof. Non-limiting examples of proteasome inhibitors for use in the present invention include peptide aldehydes (Stein et al. WO 95/24914 published Sep. 21, 1995; Siman et al. WO 91/13904 published Sep. 19, 1991; Iqbal et al. J. Med. Chem. 38:2276-2277 (1995)), peptide boronic acids (Adams et al. WO 96/13266 published May 9, 1996; Siman et al. WO 91/13904), lactacystin, and lactacystin analogs (Fenteany et al. Proc. Natl. Acad. Sci. USA (1994) 91:3358; Fenteany et al. WO 96/32105, published Oct. 17, 1996).

Peptide aldehyde proteasome inhibitors for use in the present invention preferably are those disclosed in Stein et al. WO 95/24914 published Sep. 21, 1995 or Siman et al. WO 91/13904 published Sep. 19, 1991, both hereby incorporated by reference in their entirety.

Boronic acid or ester compounds for use in the present invention preferably are those disclosed in Adams et al. WO 96/13266 published May 9, 1996, or Siman et al. WO 91/13904, both of which are hereby incorporated by reference in their entirety.

More preferably, the boronic acid compound for use in the present invention is selected from the group consisting of: N-(4-morpholine)carbonyl-.beta.-(1-naphthyl)-L-alanine-L-leucine boronic acid; N-(8-quinoline)sulfonyl-.beta.-(1-naphthyl)-L-alanine-L-alanine-L-leucine boronic acid; N-(2-pyrazine)carbonyl-L-phenylalanine-L-leucine boronic acid, and N-(4-morpholine)carbonyl-[O-(2-pyridylmethyl)]-L-tyrosine-L-leucine boronic acid. Lactacystin and lactacystin analog compounds for use in the present invention preferably are those disclosed in Fenteany et al. WO 96/32105, published Oct. 17, 1996, hereby incorporated by reference in its entirety. More preferably, the lactacystin analog is selected from lactacystin, clasto-lactacystin .beta.lactone, 7-ethyl-clasto-lactacystin .beta.-lactone and 7-n-propyl-clasto-lactacystin .beta.-lactone are used for the methods of the invention. Most preferably the lactacystin analog is 7-n-propyl-clasto-lactacystin .beta.-lactone. The agents disclosed herein may be administered by any route, including intradermally, subcutaneously, orally, intraarterially or intravenously. Preferably, administration will be by the intravenous route. Preferably parenteral administration may be provided in a bolus or by infusion. The invention further provides an isolated and purified Jade-1 having an amino acid sequence represented by SEQ ID NO: 5, as well as fragments or derivatives thereof. Preferably functional fragments. Preferably the fragments do not include the nucleic acids encoding peptide segments consisting of just amino acids 75-83; 182-201; 217-224; 225-245; 276-288; 332-340; 343-350; 354-362; 433-439; 441-446; and 447-482 of the KIAA0215 gene with a nucleotide accession number D86969 or 107-121; 135-151; 249-264 of the KIAA0239 gene with accession number gi27529704 (Entrez database at http://www.ncbi.nlm.nih.gov/entrez/query.fcgi?CMD=&DB=nucleotide). In one preferred embodiment, the fragments do no include nucleic acids encoding 1-362 of the clone KIAA1807 with a nucleotide accession number AB058710.

In one preferred embodiment, the Jade nucleic acid sequences of the present invention encode at least 21, preferably at least 25, 30, 50, and most preferably at lest 100, 200 consecutive amino acids of the SEQ ID NO: 5. In another preferred embodiment, the nucleic acid sequence of the present invention encode a sequence comprising at least 5 consecutive amino acids selected from amino acids 1-140 and 502-509 of SEQ ID NO: 5 and at least one functional domain encoded by the SEQ ID NO: 5.

In one embodiment, the invention provides a method of treating a disease or disorder associated with decreased expression or function of a Jade family member such as Jade-1 by providing a proteasome inhibitor in a pharmaceutically acceptable carrier to an individual in need thereof and observing, for example, the Jade-1 expression level to determine the efficacy of the treatment wherein increase in the Jade-1 protein expression indicates an effective proteasome inhibitor dosage. The dose of a proteasome inhibitor is preferably determined to be effective when the cellular Jade expression is about the same or higher than in a normal tissue from the same origin. For example, when an individual with renal cancer is treated with proteinase inhibitors, the Jade expression after treatment is compared to non-cancerous renal cells taken before the treatment. Examples of diseases or disorders that may have decreased expression or function of Jade family members such as Jade-1 and can be treated with proteasome inhibitors include cancer, particularly renal cancer, VHL and renal cystic disease, particularly autosomal dominant polycystic kidney disease type I (ADPKD-1) caused by polycystin-1 mutations.

In another embodiment, the invention provides a method of decreasing Jade family expression such as Jade-1 expression in an individual wherein decreased apoptosis is desired, for example by providing a Jade inhibitor to a subject in need of treatment. Such inhibitors include, antibodies against Jade family of proteins or an immunogenic fragment thereof, antisense oligonucleotides designed to bind at least about 10-15 consecutive nucleotides in an mRNA encoding a member of a Jade family of protein or a functional fragment thereof, or an interference RNA (iRNA) designed to interfere with an mRNA encoding a member of a Jade protein family or a functional fragment thereof.

The terms “Jade protein” and “Jade family of protein” and “Jade” are used interchangeably and are meant to include Jade-1 and its homologues as described elsewhere in this specification.

The term “functional fragment” when referring to the Jade family of protein means proteins or polypeptides which retain in one designated area essentially the same biological function or activity as at least one domain of the wild type protein of SEQ ID NO:5. The terms “function” or “activity” in the methods provided by the present invention generally refers to any one of the functions of the protein comprising amino acids of SEQ ID NO: 5 and include Jade-1 induced modification of target proteins, apoptosis induction in cancer cells, induction of epithelial or neuronal differentiation, enhancing cell cycle, and interaction with at least VHL protein. For example, to be determined “functional” the Jade-1 and its functional fragments or derivatives maintain at least about 50% of the activity of the protein of SEQ ID NO:5 or a functional fragment thereof, preferably at least 75%, more preferably at least about 95% of the activity of the protein of SEQ ID NO:5 or a functional fragment thereof. This activity can be measured by use of measuring apoptosis and determining neuronal or epithelial differentiation.

For example, neuronal differentiation can be measured by culturing embryonal stem cells or neuronal stem cells and observing the appearance of any of the known neuronal differentiation markers such as N-tubulin (neuronal tubulin) against which antibodies are commercially available (e.g. Anti-Tubulin, Neuronal, clone 2G10, Upstate USA Inc., Charlottesville, Va.). Epithelial differentiation can be determined, for example by using CD44v6 and casein as markers for differentiated epithelial cells.

For example an assay measuring an increase in apoptosis is described in the examples below. For instance, cells, for example 293 cells, are transfected with a vector comprising the functional domain of interest. A specific apoptosis marker or indicator, for example Hoechst 33342, is selected and the apoptosis of the transfected cells is detected. Alternatively, the mRNA or protein level of genes expressed in apoptotic cells, such as Bcl2, can be measured using methods known in the art. The cells transfected with a vector comprising the complete Jade-1 coding sequence are used as a positive control and cells transfected with a vector without Jade-1 coding sequence are used as negative controls.

In one embodiment, the invention provides an assay for identifying agents capable of modifying Jade activity by administering to a cell expressing Jade a test agent and measuring changes in the protein modification affected by Jade. [Examples of protein modifications include acetylation, N— and O-methylation, ubiquitination, and hydroxylation. Methods of measuring such modification a re known to one skilled in the art.]

“Fragments” or “derivatives” as the terms are used herein can include competitors or peptidomimetics of the native Jade-1 with respect to a particular Jade-1 domain activity. However, the fragment or derivative shows an overall similarity to Jade-1 in other areas as explained herein. Derivatives can include agonists and antagonists derived from SEQ ID NO:5, for example by using deletion analysis to remove one or more domains or amino acids.

A Jade-1 fragment or derivative useful according to the present invention may be (i) a peptide in which one or more of the amino acid residues are substituted with a conservative or non-conservative amino acid residue (preferably a conserved amino acid residue) and such substituted amino acid residue may or may not be one encoded by the genetic code, or (ii) a peptide in which one or more of the amino acid residues includes a substituent group, or (iii) a peptide in which the mature protein is fused with another compound, such as a compound to increase the half-life of the polypeptide (for example, polyethylene glycol). For example, Jade-1 may be used to form a fusion protein with, e.g., an immunoglobulin.

A Jade protein fragments and derivatives of the invention should be of sufficient length to uniquely identify a region of Jade-1. Jade-1 fragments and derivatives thus preferably comprise at least 6 amino acids, preferably at least about 8 amino acids, usually at least about 12 amino acids, more usually at least about 15 amino acids. In the most preferred embodiment, the Jade protein fragments are at least 21, 50 or 100 amino acids long. Preferred Jade-1 fragments or derivatives of the invention include those that have at least about 55 percent homology (sequence identity) to the corresponding portion of the protein of SEQ ID NO:5, more preferably about 65 percent or more homology to the protein of SEQ ID NO:5, still more preferably about 75, even more preferably 90 percent or more homology to the protein of SEQ ID NO:5. This can readily be determined by using any standard sequence comparison program such as BLAST or PSI-BLAST at default settings. Alternatively, protein sequences of Jade-1 can be analyzed using Geno3D (http://geno3d-pbil.ibcp.fr), an automatic web server for protein molecular modeling. Starting with a query protein sequence, the server performs the homology modeling in six successive steps: (i) identify homologous proteins with known 3D structures by using PSI-BLAST; (ii) provide the user all potential templates through a very convenient user interface for target selection; (iii) perform the alignment of both query and subject sequences; (iv) extract geometrical restraints (dihedral angles and distances) for corresponding atoms between the query and the template; (v) perform the 3D construction of the protein by using a distance geometry approach and (vi) finally send the results by e-mail to the user. (Combet et al. Bioinformatics January 2002; 18(1):213-4). A number of improvements to the existing sequence comparison programs are suggested to significantly improve the sequence alignment and homology searches are known (Elofson, Proteins Feb. 15, 2002;46(3):330-9). Homology searches are run at the standard default setting. The isolated and purified fragments of Jade-1 of the present invention preferably exclude the specific nucleotide fragments published in the clone KIAA1807 (Nagase et al. DNA Research 8:85-95, 2001). The preferred nucleic acid sequences used are 6000 nucleotides or less, more preferably 3000 nucleotides or less in length.

The homologous proteins and nucleic acids useful according to the treatment and diagnostic methods of the present invention include clone KIAA0215, which is an 823 amino acid protein that is 59% identical and 74% similar to Jade-1 amino acids 1-502 and clone KIAA0239, which is also 79% similar to Jade-1 starting from the first PHD domain onward for 283 amino acids. There are 286, 498, and 577 aa 850 alternatively spliced versions of clone KIAA0239. In the Celera database, a 766 amino acid clone from amino acids 31 to 454 is 65% identical and 75% similar to Jade-1 amino acids 51 to 502. The homologous nucleic acids further include a Drosophila homolog CG7036 which contains 3201 amino acids, but over aas 109 to 599 has 41% identity 57% similarity to Jade-1 amino acids 8 to 502. Sequence analysis of this Drosophila clone beyond the Jade-1 homology region reveals almost no homologies by BLAST search, but does identify a high-scoring WSC B domain at amino acids 783 to 829 (E-value 0.00064) by BLOCK search.

WSC proteins contain 3 characteristic domains (A, B, and C) that are thought to bind carbohydrates and, interestingly, all of which are found in the PKD1 family of proteins. WSC proteins are transmembrane proteins involved in maintaining cell wall integrity and contributing to stress response (heat shock) in yeast (Verna-J et al PNAS 97). Moreover, yeast WSC proteins present in the secretory path are required for arrest of the secretory response (ASR), suggesting that accumulated WSC proteins initiate the ASR (Nanduri-J Mol Cell 2001). WSC2p is the family member required for the ASR. This ASR pathway is also distinct from ER stress-related unfolded protein response. The effect of the WSC family in ASR may be mediated by PKC1-MPK1 (Verna, Nanduri). C. elegans 744 amino acid AAF59546 protein from amino acids 138 to 508 is 46% similar to Jade-1 over amino acids 71 to 458. Little additional homology is identifiable beyond the Jade-1 related region by BLOCKs or BLAST searches. Drosophila clone CG1845 is a 1430 amino acid weaker homolog (47% similarity) over Jade-1 amino acids 136 to 501. CG1845 contains a bromodomain (aas 646 to 691), a strong probable PWWP A domain (amino acids 1309 to 1323) and also a possible WSC C domain (amino acids 930 to 951) with BLOCKs E-value of 0.056. Other Jade-1 homologs contain bromodomains. For example the 1214 amino acid protein Br140 or Peregrin, which maps to chromosome 3p25, over amino acids 210 to 600 is 44% similar to Jade-1 amino acids 135 to 501.

Other related bromodomain-containing proteins include BRL and BRF3 (KIAA1286), which appears to be related or the same as a different Br140 splice. Several of the genes involved in acute myelogenous leukemia, such as MLL (human ortholog of Drosophila trithorax), AF10, AF17 have high homology to the Jade-1 PHD region, including PHD1 PHD2 and the interPHD region. The latter with PHD2 has also been given a designation of extended PHD or extended LAP domain. Similar PHD/extended PHD features are found in human clones KIAA0783, KIAA0780, KIAA0876 and KIAA0677, Drosophila Alhambra and trithorax, and C. elegans LIN49, CEZF, Saccharomyces cerevisiae Nto1p and Schizosaccharomyces pombe T39715.

Also, the sequence of the clone KIAA1807 is a useful sequence in the diagnostic and treatment methods of the present invention. The homologues described herein are also useful in producing antibodies for the diagnostic and/or treatment methods described elsewhere in the specification.

The Jade homologues as described above are also useful in the assays to identify inhibitors or enhancers for Jade family of proteins wherein the nucleic acid sequences encoding these Jade homologues or fragments thereof can be substituted for the SEQ ID NO: 5 in the methods described elsewhere in the specification.

The plant homeodomain (PHD) was first identified in proteins involved in patterning in plants. They have since proved to have important regulatory roles in development, cancer biology, and immunity. The PHD may participate directly in transcriptional activation (AIRE) (47, 48), repression (KAP) (49), DNA binding (HAT3.1) (50), or chromatin localization (ATRX) (51). Like the related RING-type zinc finger, a PHD motif may possess ubiquitin ligase activity (52). By analogy, the PHD may also be required for ligation of small ubiquitin-like molecules. PHD proteins may be acetyltransferases with the PHD being a key motif required for this activity (CBP/p300) (53). Jade-1 may have one or more of these functions and may act both in the nucleus and cytoplasm to carry them out.

Interestingly, leucocytes carrying mutations/translocations in AF10 and AF17 genes are leukemic and are unable to terminally differentiate. Other genes such as MOZ tumor suppressor gene and p300/CBP family of transcriptional co-activators, that also contain a PHD domain, have been reported as being involved in histone acetylation (Olufunmilayo et al. Chapter 6 in Cancer Medicine e5, pp. 88-106 at http://www.cancer.org/downloads/PUB/DOCS/SECTION1/6.pdf).

All of the above discussed proteins are considered as exemplary members of the Jade family and are consequently useful in the treatment and diagnostic methods of the present invention. Other members of the family may be subsequently identified. The members of the Jade family will have at least about 20% amino acid similarity over a conserved Jade protein domain, such as a single Jade PHD region.

Jade-1 and fragments and derivatives thereof of the invention are “isolated”, meaning the protein or peptide is not in its natural cellular environment. For example, it is about 70% free of immunoglobulin contaminants, more preferably at least 85% free, still more preferably at least 90% free and even more preferably at least 95% free of immunoglobulin contaminants. The Jade-1 fragments and derivatives may be present in a free state or bound to other components, e.g. blocking groups to chemically insulate reactive groups (e.g. amines, carboxyls, etc.) of the peptide, or fusion peptides or polypeptides (i.e. the peptide may be present as a portion of a larger polypeptide).

Preferably, the protein, peptides or nucleic acids of the present invention are at least about 85% pure, still more preferably at least 90% pure, even more preferably at least 95% pure, and still more preferably at least 98% pure.

Jade-1 protein of the present invention is a short-lived PEST- and plant homeodomain (PHD)-containing protein that resides, for example, in the nuclear speckles in renal proximal tubule cells, which are clear-cell renal cancer precursors. Further, according to our data from the Northern blot hybridization analysis, Jade-1 mRNA appears to be expressed ubiquitously. However, the protein expression levels between different tissues vary significantly suggesting that the regulation of Jade expression occurs at the protein level. The strongest expression of Jade protein is found in kidney then brain, muscle, heart and liver. In addition to renal cancer cell lines, we further identified expression of Jade-1 in colon cancer cell line and showed that the expression of Jade-1 also in these cells was inducible with proteasome inhibitors. The over expresion of Jade and members of the family is indicitive of cancer. In those cells the regulation results in increased apoptosis.

Jade-1 exhibits strong expression in kidney and positive regulation by proximal tubule cell differentiation and by wild-type VHL and mutant VHL proteins that do not cause renal cancer, but not renal cancer-causing VHL mutations. Jade-1 inhibits cancer cell growth and causes apoptosis that can be blocked by wild-type, but not mutated VHL.

Jade-1 maps to chromosome 4q28, a region commonly deleted in renal cancer. Jade-1 is distinct from but functionally reminiscent of Ing1, a smaller PHD protein upregulated with apoptosis that is a p53 transcriptional coactivator of p21. Ing1 harbors PHD region mutations in squamous cell cancers and is a good candidate tumor suppressor. Ing1 has been recently shown to be involved in histone acetylation (Vieyra et al. J Biol Chem 277:29632-29639, 2002).

Jade-1 participates in a VHL-mediated renal tumor suppressor pathway, plays a direct role in VHL disease, ADPKD and renal cancers. Jade-1, its homologues and agonists or molecules that affect the Jade cellular pathways are therefore potential therapeutic agents in VHL disease lesions, renal cancer and other malignancies.

Jade-1 is the first member of a family of proteins that have not been previously characterized. Other Jade family members, which are discussed above, likely exhibit similar anti-tumor or proapoptotic qualities and are therefore themselves candidate tumor suppressors, disease genes, and therapeutic molecules and/or targets. Identification of Jade family genetic mutations or changes in expression may have diagnostic importance in renal cancer, renal disease, other cancers, and other diseases having abnormalities in the cell cycle or apoptotic pathways.

As discussed above, Jade-1 nucleic acid fragments and derivatives are also provided. Those fragments and derivatives are of a length sufficient to bind to the sequence of SEQ ID NO:4 or 7 under the following moderately stringent conditions (referred to herein as “normal stringency” conditions): use of a hybridization buffer comprising 20% formamide in 0.8M saline/0.08M sodium citrate (SSC) buffer at a temperature of 37° C. and remaining bound when subject to washing once with that SSC buffer at 37° C.

Preferred Jade-1 nucleic acid fragments and derivatives of the invention will bind to the sequence of SEQ ID NO:2 under the following highly stringent conditions (referred to herein as “high stringency” conditions): use of a hybridization buffer comprising 20% fonnamide in 0.9M saline/0.09M sodium citrate (SSC) buffer at a temperature of 42° C. and remaining bound when subject to washing twice with that SSC buffer at 42° C.

These nucleic acid fragments and derivatives preferably should comprise at least 17 base pairs, more preferably at least 20 base pairs, still more preferably comprise at least 25 base pairs, even more preferably at least about 30 base pairs. In some preferred embodiments, the nucleic acid fragment or derivative is bound to some moiety which permits ready identification such as a radionucleotide, fluorescent or other chemical identifier.

Isolated Jade-1 and peptide fragments or derivatives of the invention are preferably produced by recombinant methods. A wide variety of molecular and biochemical methods are available for generating and expressing the Jade-1 of the present invention; see e.g. the procedures disclosed in Molecular Cloning: A Laboratory Manual (3rd Ed., Sambrook and Russel, Cold Spring Harbor Laboratory Press, 2001), Current Protocols in Molecular Biology (Eds. Ausubel, Brent, Kingston, More, Feidman, Smith and Stuhl, Greene Publ. Assoc., Wiley-Interscience, NY, N.Y. 1992) or other procedures that are otherwise known in the art. For example, Jade-1 or fragments thereof may be obtained by chemical synthesis, expression in bacteria such as E. coli and eukaryotes such as yeast, baculovirus, or mammalian cell-based expression systems, etc., depending on the size, nature and quantity of the Jade-1 or fragment. The use of mammalian-based expression systems, particularly human, is particularly preferred where the peptide is to be used therapeutically.

Nucleic acids encoding the novel Jade-1 of the present invention and fragments and derivatives thereof may be part of Jade-1 expression vectors and may be incorporated into recombinant cells for expression and screening, transgenic animals for functional studies (e.g. the efficacy of candidate drugs for disease associated with expression of a Jade-1), etc. Nucleic acids encoding Jade-1 containing proteins are isolated from eukaryotic cells, preferably human cells, by screening cDNA libraries with probes or PCR primers derived from the disclosed Jade-1 cDNAs.

The nucleic acids of the present invention are isolated, meaning that the nucleic acids comprise a sequence joined to a nucleotide other than that which it is joined to on a natural chromosome and usually constitutes at least about 0.5%, preferably at least about 2%, and more preferably at least about 5% by weight of total nucleic acid present in a given fraction. A partially pure nucleic acid constitutes at least about 10%, preferably at least about 30%, and more preferably at least about 60% by weight of total nucleic acid present in a given fraction. A pure nucleic acid constitutes at least about 80%, preferably at least about 90%, and more preferably at least about 95% by weight of total nucleic acid present in a given fraction.

The nucleic acids of the present invention find a wide variety of applications including: use as translatable transcripts, hybridization probes, PCR primers, therapeutic nucleic acids, etc.; use in detecting the presence of Jade-1 genes and gene transcripts; use in detecting or amplifying nucleic acids encoding additional Jade-1 homologs and structural analogs; and use in gene therapy applications and use for production of antigens for antibody production. PCR primers and hybridization probes are useful in diagnostic methods provided as one embodiment of the present invention. The nucleic acid sequences of the present invention are also useful in designing antisense oligonucleotides to inhibit Jade expression.

To enhance Jade-1 activity, nucleic acid encoding a constitutively expressed Jade-1 can be administered to a subject. One preferred embodiment employs nucleic acid encoding a Jade-1 derivative that restores the Jade-1 production to cells where Jade-1 is either not expressed or where it is expressed in an inactive form. For example, in cancer many apoptosis inducing reactions are desirable, and it is therefore advantageous to activate those activities dependent upon Jade-1 activity.

Accordingly the wild-type Jade gene or a functional part of the gene such as a domain supplying a particular function may be introduced into the cell in a vector such that the gene remains extrachromosomal. In such a situation, the gene will be expressed by the cell from the extrachromosomal location.

Ing1, or Inhibitor of Growth (62), is a p53 binding protein and good candidate tumor suppressor (63). Ing1 is a single PHD containing 33 kDa protein upregulated with UV-induced apoptosis (61) and cell senescence (64). Ing1 binds p53 via a non-PHD region and enhances p53 transcription of p21 (63), a key cell cycle inhibitor. In addition to chromosomal deletions at the ing1 locus at 13q33-34, ing1 mutations affecting the PHD domain have been found in squamous cell cancers of the head and neck (50) and esophagus (51), supporting its role as a tumor suppressor.

If a Jade-1 encoding nucleic acid is introduced and expressed in a cell carrying a mutant Jade gene, the gene portion should encode a part that is defective or deficient in that cell, e.g. domain that is important for the apoptosis pathway. More preferred is the situation where the wild-type tumor suppressor pathway gene or a part of it is introduced into the mutant cell in such a way that it recombines with the endogenous mutant Jade gene present in the cell. Such recombination would require stable integration into the cell such as via a double recombination event that would result in the correction of the gene mutation.

Vectors for introduction of genes both for recombination and for extrachromosomal maintenance are known in the art and any suitable vector may be used. Such a cell can be used in a wide range of activities. For example, one can prepare a drug or therapeutic agent screen using a tumor cell line having a defect in the tumor suppressor pathway and by this technique create a control cell from that tumor cell. Thus, one can determine if the compounds tested affect the tumor suppressor pathway by contacting said cell with the candidate agents and detecting whether Jade expression is increased compared to the control as a result of adding the agent. Such a method can be used to select drugs that specifically affect the pathway or as a screen for agents, including known anti-cancer agents that are effective against the tumors wherein Jade expression is decreased. These drugs may be combined with other drugs for their combined or synergistic effects.

Alternatively, to inhibit Jade-1 activity, nucleic acids capable of inhibiting translation of Jade-1 also may be administered. These nucleic acids are typically antisense: single-stranded sequences comprising complements of the disclosed relevant Jade-1 fragment-encoding nucleic acid. Antisense modulation of the expression of a given Jade-1 fragment containing protein may employ Jade-1 fragment antisense nucleic acids operably linked to gene regulatory sequences. Cells are transfected with a vector comprising a Jade-1 fragment sequence with a promoter sequence oriented such that transcription of the gene yields an antisense transcript capable of binding to endogenous Jade-1 fragment containing protein encoding mRNA. Transcription of the antisense nucleic acid may be constitutive or inducible and the vector may provide for stable extrachromosomal maintenance or integration. Alternatively, single-stranded antisense nucleic acids that bind to genomic DNA or mRNA encoding a given Jade-1 fragment containing protein may be administered to the target cell, in or temporarily isolated from a host, at a concentration that results in a substantial reduction in expression of the Jade-1. For example, we have used a synthetic modified oligonucleotide complementary to the first 20 Jade-1 coding nucleotides 5′-GGAAGGCGACCTCGTTTCAT-3′ (SEQ ID NO: 6) to reduce endogenous Jade-1 protein levels in cultured cells.

The Jade-1 nucleic acids are introduced into the target cell by any method that will result in the uptake and expression of the nucleic acid by the target cells. These can include vectors, liposomes, naked DNA, adjuvant-assisted DNA, gene gun, catheters, etc. Vectors include chemical conjugates such as described in WO 93/04701, which has targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic.

Preferred vectors include viral vectors, fusion proteins and chemical conjugates. Retroviral vectors include moloney murine leukemia viruses and lentiviral vectors. DNA viral vectors are preferred. These vectors include pox vectors such as orthopox or avipox vectors, herpesvirus vectors such as a herpes simplex I virus (HSV) vector (Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.:90 7603 (1993); Geller, A. I., et al., Proc Natl. Acad. Sci USA: 87:1149 (1990)), Adenovirus Vectors (LeGal LaSalle et al., Science, 259:988 (1993); Davidson, et al., Nat. Genet 3: 219 (1993); Yang, et al., J. Virol. 69: 2004 (1995)), Adeno-associated Virus Vectors (Kaplitt, M. G., et al., Nat. Genet. 8:148 (1994)) and gutless adenovirus vectors (Ferry N. et al. Hum Gene Ther Sep. 20, 1998; 9(14):1975-81).

Pox viral vectors introduce the gene into the cell's cytoplasm. Avipox virus vectors result in only a short-term expression of the nucleic acid. Adenovirus vectors, adeno-associated virus vectors and herpes simplex virus (HSV) vectors are preferred for introducing the nucleic acid into neural cells. The adenovirus vector results in a shorter-term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaPO₄ precipitation, DEAE dextran, electroporation, protoplast fusion, lipofecton, cell microinjection, and viral vectors.

The vector can be employed to target essentially any desired target cell, such as a glioma. For example, stereotaxic injection can be used to direct the vectors (e.g. adenovirus, HSV) to a desired location. Additionally, the particles can be delivered by intracerebroventricular (icv) infusion using a minipump infusion system, such as a SynchroMed Infusion System. A method based on bulk flow, termed convection, has also proven effective at delivering large molecules to extended areas of the brain and may be useful in delivering the vector to the target cell (Bobo et al., Proc. Natl. Acad. Sci. USA 91:2076-2080 (1994); Morrison et al., Am. J. Physiol. 266: 292-305 (1994)). Other methods that can be used include catheters, intravenous, parenteral, intraperitoneal and subcutaneous injection, and oral or other known routes of administration.

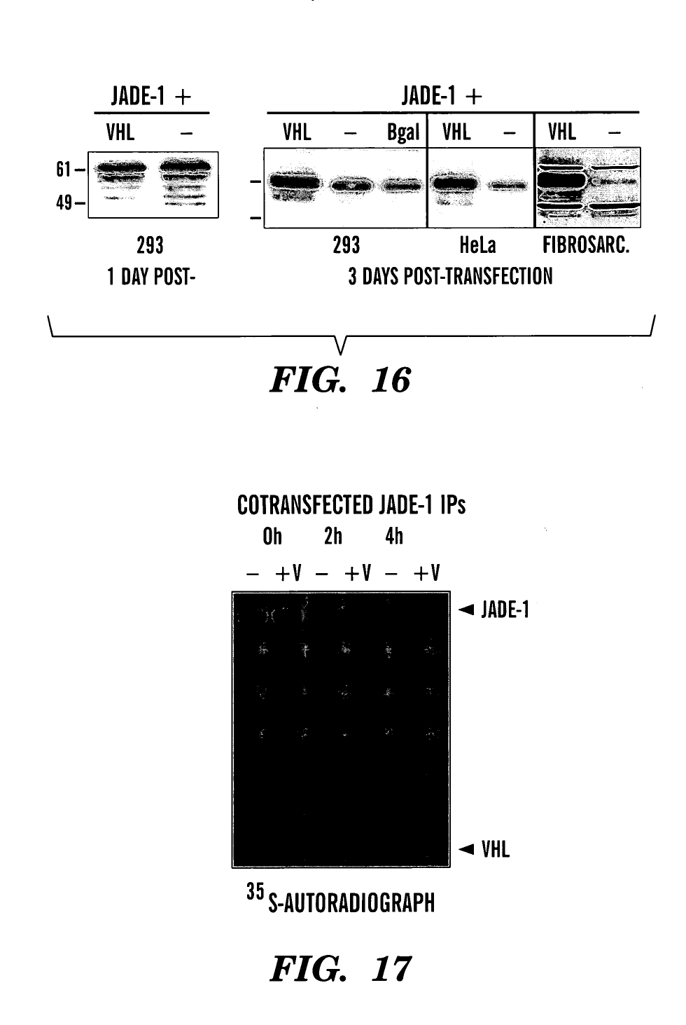

Based upon co-localization data, at most 10% of VHL associates with Jade-1. No purification studies have been previously performed on kidney tissue where Jade-1 is most highly expressed and where the interacting fractions of Jade-1 and VHL are the largest. Moreover, 786-O cells, where most co-immunoprecipitation work has been done, express little Jade-1 unless they are grown to late confluence (FIG. 16). We also discovered that the half-life of Jade-1 protein is fairly brief.

Many genes regulated by VHL are really regulated transcriptionally by HIF (12) and so are downregulated by VHL. VHL is a known tumor suppressor and it is known to bind transcription factors elongin B, elongin C and cullin-2, proteins that are associated with transcriptional elongation and ubiquitination (Tyers and Willems, Science 284:602-604, 1999). Because addition of wildtype VHL to VHL negative 786-O renal cell carcinoma cell line (RCC) restores cells' ability to exit the cell cycle, VHL also seems to be involved in cell cycle regulation (Pause et al. Proc Natl Acad Sci USA 95:993-998, 1998). VHL is also known to regulate directly the hypoxia-inducible factor (HIF) (Maxwell et al. Nature 399:271-275, 1999) by ubiquitination and is associated with microtubules protecting them from depolymerization (Hergovich et al. Nature Cell Biol 5:64-70, 2003). In addition, like HIF (12), hnRNP A2 (20) and PKC lambda (70) are degraded via VHL-mediated ubiquitination and therefore also downregulated by VHL. In contrast, VHL directly binds fibronectin protein and increases its abundance and incorporation into extracellular matrix, perhaps through the endoplasmic reticulum (ER) and/or Golgi (30). VHL upregulates the cell cycle inhibitor p27 (31, 31, 71). VHL also promotes NEDDylation of cul-2, which may stabilize cul-2 in addition to increasing its ubiquitinating activity (72, 73). Thus, a precedent exists for VHL to increase protein expression.

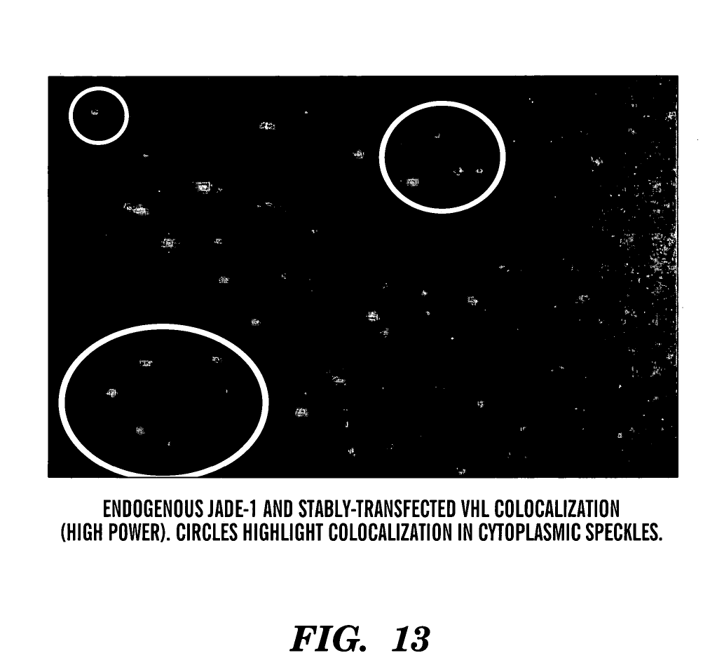

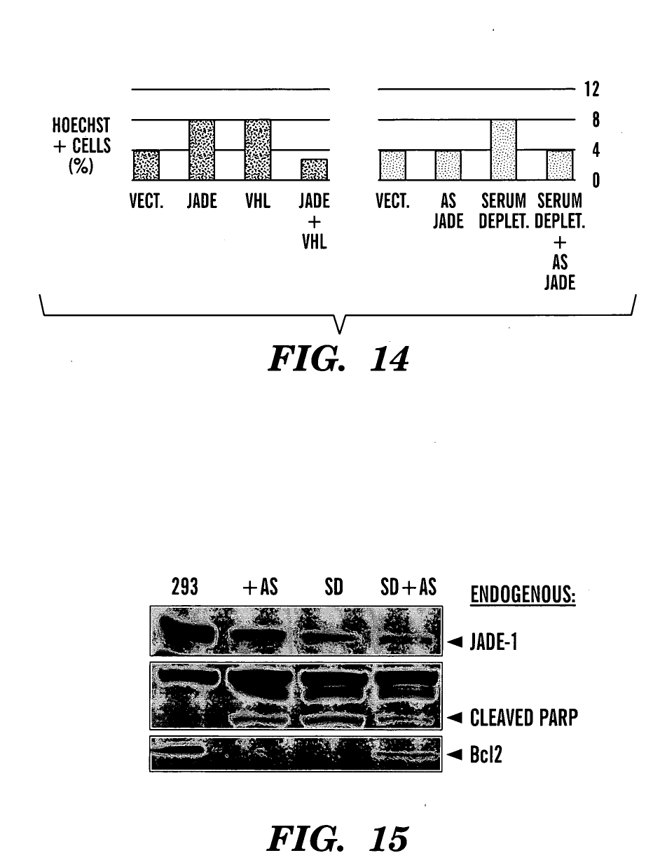

In fact, the cytoplasmic speckle co-localization of Jade-1 and VHL (FIG. 13) may be in ER or Golgi, so the mechanism should be similar to fibronectin's, because only 10% of VHL and Jade-1 associate in renal cancer cells, a “hit-and-run” interaction is supported in which VHL might induce a stabilizing Jade-1 modification, perhaps affecting proteasomal degradation. Moreover, endogenous (FIG. 15) and transfected Jade-1 both appear as a close doublet, with the larger band having the longer half-life in metabolic labeling experiments (FIG. 15), suggesting VHL might promote a stabilizing Jade-1 post-translational modification.

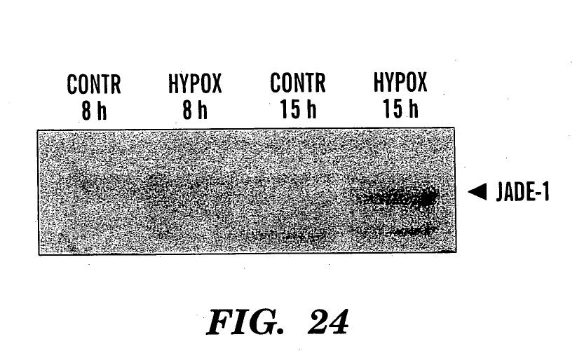

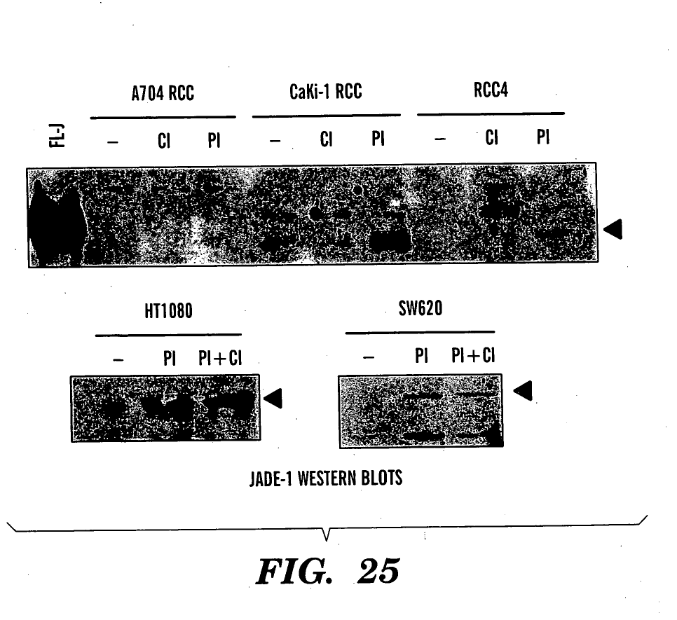

The Jade proteins likely have many cellular functions. The results above indicate the Jade family of proteins are likely to be involved in cell stress response (FIGS. 23 and 24), perhaps sensing or contributing to hypoxic stress, ER stress, ASR stress, DNA damage stress, growth factor withdrawal, or other cellular stress. This family of proteins may also directly or indirectly signal, perhaps in situ via a cascade or by transcriptional control of gene expression, to initiate a cell death program. Protein modification is also one possible mechanism how the Jade family of proteins regulate cell stress responses.

The present invention provides efficient screening methods to identify pharmacological agents or lead compounds for agents that modulate, e.g. interfere with or increase Jade-1 activity. The methods are amenable to automated, cost-effective high throughput drug screening and have immediate application in a broad range of pharmaceutical drug development programs.

A wide variety of assays for Jade family binding agents, preferably Jade-1 binding agents are provided including, e.g., labeled in vitro protein-protein binding assays, electrophoretic mobility shift assays, immunoassays, cell based assays such as one, two and three hybrid screens and expression assays.

A binding assay mixture of the invention comprises at least a portion of a Jade family protein, such as the Jade-1 protein. An assay mixture of the invention also comprises a candidate pharmacological agent. Generally a plurality of assay mixtures are run in parallel with different candidate agent concentrations to obtain a differential response to the various concentrations. Typically, one of these assay mixtures serves as a negative control, i.e. at zero concentration or below the limits of assay detection. Candidate agents encompass numerous chemical classes, though typically they are organic or inorganic compounds and preferably small organic compounds. Small organic compounds generally have a molecular weight of more than about 50 yet less than about 2,500. Candidate agents comprise functional chemical groups necessary for structural interactions with proteins and/or DNA, and may include at least one or two amine, carbonyl, hydroxyl or carboxyl groups.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced.

Additionally, natural and synthetically produced libraries and compounds are readily modified through conventional chemical, physical, and biochemical means. In addition, known pharmacological agents may be subject to directed or random chemical modifications, such as acylation, alkylation, esterification, amidification, etc.

A variety of other reagents may also be included in the mixture. These include reagents such as salts, buffers, neutral proteins, e.g. albumin, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

The resultant mixture is then incubated under conditions whereby the candidate pharmacological agent and the Jade family member such as Jade-1 or fragment or derivative thereof, if capable, bind. The mixture components can be added in any order that provides for the requisite bindings. Incubations may be performed at any temperature that facilitates optimal binding, typically between 4° and 40° C., more commonly between 15° and 40° C. Incubation periods are likewise selected for optimal binding but also minimized to facilitate rapid, high throughput screening, and are typically between 0.1 and 10 hours, preferably less than 5 hours, more preferably less than 2 hours.

After incubation, the binding is detected by any convenient way. For cell-free type assays, the member, for example Jade-1, may be bound to a solid substrate and the agent labeled, e.g., radiolabeled. A separation step can be used to separate the bound Jade-1 from unbound agent. The separation step may be accomplished in a variety of ways known in the art. The solid substrate may be made of a wide variety of materials and in a wide variety of shapes, e.g. microtiter plate, microbead, dipstick, resin particle, etc. The substrate is chosen to maximize signal to noise ratios, to minimize background binding, to facilitate washing and to minimize cost.

Separation may be effected for example, by removing a bead or dipstick from a reservoir, emptying or diluting a reservoir such as a microtiter plate well, rinsing a bead (e.g. beads with iron cores may be readily isolated and washed using magnets), particle, chromatographic column or filter with a wash solution or solvent. Typically, the separation step will include an extended rinse or wash or a plurality of rinses or washes. For example, where the solid substrate is a microtiter plate, the wells may be washed several times with a washing solution, which typically includes those components of the incubation mixture that do not participate in specific binding such as salts, buffer, detergent, nonspecific protein, etc. may exploit a polypeptide specific binding reagent such as an antibody or receptor specific to a ligand of the polypeptide.

As mentioned, detection may be effected in any convenient way, and for cell-free assays, one of the components usually comprises or is coupled to a label. Essentially any label can be used that provides for detection. The label may provide for direct detection as radioactivity, luminescence, optical or electron density, etc. or indirect detection such as an epitope tag, an enzyme, etc. The label may be appended to a reagent or incorporated into the peptide structure, e.g. in the case of a peptide reagent, a methionine residue comprising a radioactive isotope of sulfur.

A variety of methods may be used to detect the label depending on the nature of the label and other assay components. For example, the label may be detected bound to the solid substrate or a portion of the bound complex containing the label may be separated from the solid substrate, and thereafter the label detected. Labels may be directly detected through optical or electron density, radiative emissions, nonradiative energy transfers, etc. or indirectly detected with antibody conjugates, etc. For example, in the case of radioactive labels, emissions may be detected directly, e.g. with particle counters or indirectly, e.g. with scintillation cocktails and counters.

The assays of the invention are particularly suited to automated high throughput drug screening. In a particular embodiment, an automated mechanism, e.g. a mechanized arm, retrieves and transfers a microtiter plate to a liquid dispensing station where measured aliquots of each of an incubation buffer and a solution comprising one or more candidate agents are deposited into each designated well. The arm then retrieves and transfers to and deposits in designated wells a measured aliquot of a solution comprising a Jade-1 protein or fragment or derivative thereof as well as solutions of other reagents. Thereafter, the arm transfers the microtiter plate to an analysis station where the reaction mixture can be analyzed for the presence or absence of binding.

In one embodiment the invention provides antibodies against Jade proteins or antigenic fragments thereof. Such antibodies can readily be obtained by using antigenic portions of the protein to screen an antibody library such as a phage display library. Antibodies also can be prepared that will bind to one or more particular domains of a peptide of the invention and can be used to modulate a Jade family activity such as a Jade-1 activity. For example, we have made a rabbit antiserum against the C-terminal 20 amino acids of the human Jade-1 64 kDa protein (see below). As noted in detail above, this antibody gives a strong, readily recognizable Jade-1 signal in Western analysis for human and mouse Jade-1 and performs extremely well in immunoprecipitations of endogenous human Jade-1 and the transfected human protein. Affinity purified against the immunizing peptide, it gives a specific signal in immunocytochemistry and immunohistochemistry in human material.

QLFTHLRQDLERVMIDTDTL (SEQ ID NO: 1) C-terminal human Jade-1 peptide was used to immunize rabbits. Because of the apparent species differences in nucleotide and amino acid sequence of the 64 kDa Jade-1 between human and mouse, we immunized rabbits against two additional human Jade-1 peptides, both of which appear identical in mouse Jade-1. By BLAST search, these peptides are also unique to Jade-1 with very limited homologies to other proteins. Therefore they will give greater specificity in Western blotting and in immunohistologic and immunocytochemical studies and will be useful for both human and mouse Jade-1. The N-terminal peptide (below) is found only in 64 kDa human and mouse Jade-1 and the antiserum should therefore only recognize 64 kDa human and mouse Jade-1. The C-terminal region peptide is found in both human and mouse 95 kDa, 64 kDa, and smaller Jade-1 forms and the antiserum should therefore detect most human and mouse members of the Jade-1 group of proteins. Each of these peptides was coupled to a C-terminal cysteine for coupling to keyhole limpet hemocyanin (KLH). These peptides were specifically chosen for immunization to provide specificity for Jade-1 as well as human and mouse crossreactivity.

MKRGRLPSSSEDSDDNGSLS (SEQ ID NO: 2) N-terminal human and mouse Jade-1 peptide and NREEAHRVSVRKQKLQQ (SEQ ID NO: 3) C-terminal region of human and mouse Jade-1 peptide and similar Jade-1 peptide and antibody reagents, as well as those against the other Jade family members, including recombinant and monoclonal antibodies, will be useful in experimental work in which examination of Jade-1 protein levels are desirable. These immunologic Jade family reagents may also have additional therapeutic and diagnostic uses in the diseases outlined above, such as renal cancer, renal cystic disease, VHL disease, sporadic forms of VHL-type lesions, as well as other disease contexts not yet identified.

In one embodiment, the invention provides a kit for immunohistochemical detection of a Jade family member protein expression levels in a biological sample comprising a Jade specific antibody. The kit further comprises buffers, labels and instructions and may also comprise control biological samples to assist in diagnosis of low Jade expression levels in a tissue sample which is indicative of a condition susceptible to treatment which provides increase in Jade amount in the tissue.

VHL regulation of Jade-1, in vivo interaction of the proteins, and biologic consequences of the interaction that correlate with risk of cancer development. Coupled with the high frequency of 4q (40-45) and even 4q28 deletions (46) in renal cancer, as noted above, these observations suggest Jade-1 is a tumor suppressor and may serve as a tumor suppressor or disease gene or disease contributor or inhibitor in other contexts involving abnormal inhibition of apoptosis.

Jade family members are therapeutically useful to inhibit renal cancer or renal cyst growth because we have shown that Jade-1 inhibits growth of renal cancer cells and causes apoptosis that can be blocked by VHL. Therapeutically useful reagents include, but are not limited to, nucleic acids encoding Jade proteins or functional fragments thereof, Jade proteins or functional fragments thereof, antibodies against Jade proteins or antigenic fragments thereof, antisense molecules including modified antisense molecules, and small organic or inorganic molecules identified using the methods of the present invention all of which may be used either alone or in combination with other therapies, such as radiation or chemotherapy.

For therapeutic applications, peptides and nucleic acids of the invention may be suitably administered to a subject such as a mammal, particularly a human, alone or as part of a pharmaceutical composition, comprising the peptide or nucleic acid together with one or more acceptable carriers thereof and optionally other therapeutic ingredients. The carrier(s) must be “acceptable” in the sense of being compatible with the other ingredients of the formulation and not deleterious to the recipient thereof.

The pharmaceutical compositions of the invention include those suitable for oral, rectal, nasal, topical (including buccal and sublingual), vaginal or parenteral (including subcutaneous, intramuscular, intravenous and intradermal) administration. The formulations may conveniently be presented in unit dosage form, e.g., tablets and sustained release capsules, and in liposomes, and may be prepared by any methods well know in the art of pharmacy. See, for example, Remington's Pharmaceutical Sciences.

Such preparative methods include the step of bringing into association with the molecule to be administered ingredients such as the carrier which constitutes one or more accessory ingredients. In general, the compositions are prepared by uniformly and intimately bringing into association the active ingredients with liquid carriers, liposomes or finely divided solid carriers or both, and then if necessary shaping the product.

Compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion, or packed in liposomes and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Compositions suitable for topical administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Application of the subject therapeutics often will be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way. Systemic administration of a nucleic acid using lipofection, liposomes with tissue targeting (e.g. antibody) may also be employed.

It will be appreciated that actual preferred amounts of a given peptide or nucleic acid of the invention used in a given therapy will vary to the particular active peptide or nucleic acid being utilized, the particular compositions formulated, the mode of application, the particular site of administration, the patient's weight, general health, sex, etc., the particular indication being treated, etc. and other such factors that are recognized by those skilled in the art including the attendant physician or veterinarian. Optimal administration rates for a given protocol of administration can be readily determined by those skilled in the art using conventional dosage determination tests. In general, a suitable effective dose of one or more the above-described compounds, particularly when using the more potent compound(s), will be in the range of from 0.01 to 100 milligrams per kilogram of bodyweight of recipient per day, preferably in the range of from 0.01 to 20 milligrams per kilogram bodyweight of recipient per day, more preferably in the range of 0.05 to 4 milligrams per kilogram bodyweight of recipient per day. The desired dose is suitably administered once daily, or several sub-doses, e.g. 2 to 4 sub-doses, are administered at appropriate intervals through the day, or other appropriate schedule. Such sub-doses may be administered as unit dosage forms, e.g., containing from 0.05 to 10 milligrams of the above-described compound(s), per unit dosage, preferably from 0.2 to 2 milligrams per unit dosage.

The present invention further includes a kit for the in vivo systemic introduction of a recombinant Jade family member such as Jade-1 including fragments and derivatives thereof or nucleic acid encoding the same into a patient. Such a kit includes a carrier solution, protein or nucleic acid, and a means of delivery, e.g., a catheter or syringe. The kit may also include instructions for the administration of the preparation.

Aberrant expression of Jade can be used for diagnostic purposes. Several methods to detect increased or decreased gene expression are available to one skilled in the art. These methods include, but are not limited to RNA quantitation, protein quantitation using specific antibodies or immunohistochemica methods.

Recent comparative genomic hybridization studies indicate that a candidate renal cancer tumor suppressor resides on chromosome 4q, where Jade-1 maps. Chromosome 4q was deleted in an impressive 50% of 116 clear-cell renal cancers, whereas 3p loss occurred in 61%. Both events were clearly based on mathematical modeling (40). Similar results for 4q loss have been reported elsewhere (41, 42), and in other renal cancers (43-45). Intriguingly, 4q loss was found in 6 of 12 sarcomatoid renal cancers, with the minimum region of overlap mapping to 4q28 (46), the band that contains Jade-1 and few other genes (Unigene).

The diagnostic and prognostic methods of the present invention include looking for an alteration in mammalian Jade gene. Preferably, the mammalian Jade family gene is a human Jade-1 gene. The alteration may be due to a deletion, addition and/or mutation, such as a point mutation, in the gene. It may also be methylation. Any of these types of mutations can lead to non-functional gene products. The mutational events may occur not only in an exon, but also in an intron or non-exonic region. As a result of alterations of this kind, including alterations in non-exonic regions, effects can be seen in transcription and translation of members of the pathway, thereby affecting the ability to prevent apoptosis. The changes resulting from these alterations are also reflected in the resultant protein and mRNA as well as the gene. Other alterations that might exist in the pathway include changes that result in an increase or decrease in expression of a gene in the apoptotic pathway.

Consequently, one aspect of this invention involves determining whether there is an alteration of Jade such as Jade-1. This determination can involve screening for alterations in the gene, its mRNA, its gene products, or by detecting other manifestations of defects in the pathway. Alterations can be detected by screening for a particular apoptotic element in a suitable sample obtained, for example, from tissue, human biological fluid, such as blood, serum, plasma, urine, cerebrospinal fluid, supernatant from normal cell lysate, supernatant from preneoplastic cell lysate, supernatant from neoplastic cell lysate, supernatants from carcinoma cell lines maintained in tissue culture, eukaryotic cells, etc.

In order to detect alterations in for example Jade-1 from a particular tissue, such as a malignant tissue, it is helpful to isolate that tissue type free from the surrounding tissues. Means for enriching a tissue preparation e.g., for tumor cells, are known in the art. For example, the tissue may be isolated from paraffin or cryostat sections. Cancer cells may also be separated from normal cells by flow cytometry. These as well as other techniques for separating specific tissue types from other tissues, such as tumor from normal cells, are well known in the art. It is also helpful to screen normal tissue free from malignant tissue. Then comparisons can be made to determine whether a malignancy results from a spontaneous change in the mismatch repair pathway or is genetic.

Detection of mutations may be accomplished by cloning the Jade-1 gene present in the tissue and sequencing the gene using techniques well known in the art. For example, mRNA can be isolated, reverse transcribed and the cDNA sequenced. Alternatively, the polymerase chain reaction can be used to amplify the Jade-1 gene or fragments thereof directly from a genomic DNA preparation from the tissue such as tumor tissue. The DNA sequence of the amplified sequences can then be determined. Alternatively, one can screen for marker portions of the DNA that are indicative of changes in the DNA. The polymerase chain reaction itself is well known in the art. See e.g., Saiki et al., Science, 239:487 (1988); U.S. Pat. No. 4,683,203; and U.S. Pat. No. 4,683,195. Specific primers that can be used in order to amplify the mismatched repair genes will be discussed in more detail below.

Specific deletions of tumor suppressor pathway genes can also be detected. For example, restriction fragment length polymorphism (RFLP) probes for the Jade-1 gene or portion thereof, can be used to score loss of a wild-type allele. Other techniques for detecting deletions, as are known in the art, can be used.

Loss of the wild-type Jade-1 may also be detected on the basis of the loss of a wild-type expression product. Such expression products include both the mRNA as well as the protein product itself. Point mutations may be detected by sequencing the mRNA directly or via molecular cloning of cDNA made from the mRNA. The sequence of the cloned cDNA can be determined using DNA sequencing techniques that are well known in the art. Alternatively, one can screen for changes in the protein. For example, a panel of antibodies, for example single chain or monoclonal antibodies, could be used in which specific epitopes involved in, for example, Jade-1 apoptosis inhibiting functional domains or VHL interacting domains are represented by a particular antibody. Loss or perturbation of binding of a monoclonal antibody in the panel would indicate mutational alteration of the protein and thus of the gene itself. Alternatively, deletional mutations leading to expression of truncated proteins can be quickly detected using a sandwich type ELISA screening procedure, in which, for example, the capture antibody is specific for the C-terminal portion of the pathway protein. Failure of a labeled antibody to bind to the C-terminal portion of the protein provides an indication that the protein is truncated. Even where there is binding to the C-terminal, further tests on the protein can indicate changes. For example, molecular weight comparison. Any means for detecting altered mismatch repair pathway proteins can be used to detect loss of wild-type mismatch repair pathway genes.

Alternatively, mismatch detection can be used to detect point mutations in a Jade gene, mRNA or gene product, such as the Jade-1 gene or its mRNA product. An example of a mismatch cleavage technique is the RNAase protection method, which is described in detail in Winter et al., Proc. Natl. Acad. Sci. USA, 82:7575 (1985) and Meyers et al., Science, 230:1242 (1985). In the practice of the present invention, the method involves the use of a labeled riboprobe that is complementary to for example the human wild-type Jade-1. The riboprobe and either mRNA or DNA-isolated form the test tissue are hybridized together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full-length duplex RNA for the riboprobe and the mismatch repair pathway mRNA or DNA. If the riboprobe comprises only a segment of the Jade-1 mRNA or gene it will be desirable to use a number of these probes to screen the whole mRNA sequence for mismatches.

In similar fashion, DNA probes can be used to detect mismatches, through enzymatic or chemical cleavage. See, e.g., Cotton et al., Proc. Nat. Acad. Sci. USA, 85:4397 (1988); and Shenk et al., Proc. Natl. Acad. Sci. USA, 72:989 (1975). Alternatively, mismatches can be detected by shifts in the electrophoretic mobility of mismatched duplexes relative to matched duplexes. See, e.g., Cariello, Human Genetics, 42:726 (1988). With either riboprobes or DNA probes, the cellular mRNA or DNA that might contain a mutation can be amplified using PCR before hybridization.

DNA sequences of the family such as the Jade-1 gene from test tissue that have been amplified by use of PCR may also be screened using allele-specific probes. These probes are nucleic acid oligomers, each of which contains a region of the Jade-1 gene sequence harboring a known mutation. By use of a battery of allele-specific probes, the PCR amplification products can be screened to identify the presence of a previously identified mutation in the gene. Hybridization of allele-specific probes with amplified mismatch repair pathway sequences can be performed, for example, on a nylon filter. Hybridization to a particular probe indicates the presence of the same mutation in the tumor tissue as in the allele-specific probe.

Altered Jade genes or gene products can be detected in a wide range of biological samples, such as serum, stool, or other body fluids, such as urine and sputum. The same techniques discussed above can be applied to all biological samples. By screening such biological samples, a simple early diagnosis can be achieved for many types of abnormalities such as defects in chromosomal segregation or cancers. For example someone can be screened as part of a pre-pregnancy battery of tests. Thus, if fertility problems arise, the knowledge of the defect can be used in determining the treatment. Moreover, even if a pregnancy results, the knowledge can be used in determining whether and the types of pre-natal screening.

Similarly, even when someone has been diagnosed with cancer or other Jade related disease, these screens can be prognostic of the condition, e.g., spontaneous mutation versus hereditary. The prognostic method of the present invention is useful for clinicians so that they can decide upon an appropriate course of treatment. For example, a hereditary mutation in the tumor suppressor system may suggest a different therapeutic regimen than a sporadic mutation. Thus, knowing of a defect permits one to choose an appropriate course of therapy.

The methods of screening of the present invention are applicable to any sample in which defects in Jade genes have a role, such as in tumorigenesis.

The method of the present invention for diagnosis of, for example, a tumor suppressor defective tumor is applicable across a broad range of tumors. These include renal, breast, lung, colorectal, ovary, endometrial (uterine), bladder, skin, rectal and small bowel.

The present invention also provides a kit useful for determination of the nucleotide sequence of a Jade family member such as Jade-1 using a method of DNA amplification, e.g., PCR or an antibody. The kit comprises a set of pairs of single stranded oligonucleotide DNA primers which can be hybridized to sequences within or surrounding for example the Jade-1 gene in order to prime amplifying DNA synthesis of the gene itself or to use as antibody for the gene product. In one preferred embodiment instructions for using the materials to screen for Jade-1 for diagnosis or prognosis purposes are included.

To facilitate subsequent cloning of amplified sequences, primers may have restriction enzyme sites appended to their 5′ ends. Thus, all nucleotides of the primers are derived from the mismatch repair gene sequences or sequences adjacent thereto except the few nucleotides necessary to form a restriction enzyme site. Such enzymes and sites are well known in the art. The primers themselves can be synthesized using techniques that are well known in the art. Generally, the primers can be made using synthesizing machines that are commercially available.

In one embodiment, the set of primer pairs for detecting alterations in the Jade-1 gene comprises primer pairs that cover the complete coding region of the Jade-1 gene (SEQ ID NO: 4) or parts of the non-coding sequence presented in SEQ ID NO: 7. One can readily derive other primers to use based upon the sequences. Typically the primer will be at least about 10 nucleotides, more preferably at least about 13 nucleotides, still more preferably at least about 15 nucleotides, even more preferably at least about 20 nucleotides. Typical primer sizes will range from about 17 to 23 nucleotides.

The present invention further provides a method for determining whether an alteration in a Jade, for example Jade-1, gene is a mutation or an allelic variation. The method comprises introducing the altered gene into a cell having a mutation in the Jade gene being tested. The cell may be in vitro or in vivo. If the altered gene tested is an allelic variation, i.e., function is maintained, the mutation will be complemented and the cell will exhibit a wild-type phenotype. In contrast, if the altered gene in a mutation, the mutation will not be complemented and the cell will continue to exhibit non-wild type phenotype.

One can also prepare cell lines stably expressing Jade, for example Jade-1. Such cells can be used for a variety of purposes, for example, the cells can be used as a source of antigen for preparing a range of antibodies using techniques well known in the art.

Polypeptides or other molecules that have functional Jade activity may be supplied to cells that carry mutant alleles. The active molecules can be introduced into the cells by microinjection or by liposomes, for example. Alternatively, some such active molecules may be taken up by the cells, actively or by diffusion. Supply of such active molecules will affect a desired state, for example, apoptosis.

Predisposition to a difficulty with appropriate segregation of chromosomes or to cancers can be ascertained by testing normal tissues of humans. For example, a person who has inherited a germline Jade gene alteration would be prone to develop cancer. This can be determined by testing DNA or mRNA from any tissue of the person's body. Most simply, blood can be drawn and the DNA or mRNA extracted from cells of the blood. Alteration or loss of a wild-type Jade gene allele function or expression, either by point mutation, insertion or by deletion, can be detected by any of the means discussed above. Nucleic acid can also be extracted and tested from fetal tissues for this purpose.

We have also detected Jade-1 expressed in thymus, which contains largely T-cells, and in spleen, which comprises primarily B cells. A recent study on the autoimmune regulator protein AIRE, which is a PHD protein encoded by the gene for the autoimmune APECED syndrome, shows that this protein increases expression of non-lymphocytic tissue-restricted proteins in thymic T-cells (Anderson et al. Science. November 15, 2000;298(5597):1395-401). Thus, it has been proposed that in APECED disease the absence of AIRE protein prevents immature T-cells from seeing the rare tissue-restricted self peptides and that mature T-cells progeny promotes an inappropriate autoimmunity. Therefore, without wishing to be bound by a theory, we propose that Jade-1 or other Jade family members play similar regulatory roles in the normal immune response and in autoimmunity. Thus, the invention also provides a diagnostic, prognostic and treatment methods for autoimmune disease using detection and/or regulation of Jade family of proteins. Detecting an aberrant expression pattern of Jade family of proteins in the thymus and/or spleen using, e.g. antibodies directed against Jade family of proteins, provides a method of identifying individuals who will be responsive to treatment with Jade activity regulating treatment regimes.

Accordingly, the present invention provides for a wide range of assays (both in vivo and in vitro). These assays can be used to detect cellular activities affected by the Jade activity such as tumor suppression, which include identification of eukaryotic nucleotide sequences that are homologous to bacterial or yeast Jade and the cellular activities of the polypeptides they encode. In these assay systems, Jade genes, polypeptides, unique fragments, or functional equivalents thereof, may be supplied to the system or produced within the system. For example, such assays could be used to determine whether there is a Jade gene excess or depletion. For example, a in vivo assay systems may be used to study the effects of increased or decreased levels of transcript or polypeptides of the invention in cell or tissue cultures, in whole animals, or in particular cells or tissues within whole animals or tissue culture systems, or over specified time intervals (including during embryogenesis).

The present invention is further illustrated by the following Examples. These Examples are provided to aid in the understanding of the invention and are not construed as a limitation thereof.

### EXAMPLE

Von Hippel-Lindau (VHL) disease is a rare autosomal dominant cancer syndrome caused by a VHL gene which is located on chromosome 3p25.5 (11). The manifestations of the disease include retinal angiomas, central nervous system hemangioblastomas; renal and pancreatic cysts; pheochromocytomas; and renal cell carcinoma The variety of manifestations of the VHL suggest that the VHL protein has multiple cellular functions. In addition, many families get only some manifestations, suggesting strongly that certain VHL mutations disrupt some, but not all, VHL cellular pathways. The most malignant VHL disease related cell growth abnormality is clear cell renal cancer indicating that VHL protein exerts tumor suppressor activity in renal proximal tubules, the tumor's precursor cells. Remarkably, 75% of sporadic clear-cell renal cancers harbor biallelic VHL mutations (1, 2), indicating that the VHL gene is the major renal cancer gene in adults but that there are likely other elements that contribute to the development of the disease.

VHL has many cellular functions including oxygen sensing. VHL directly binds and targets transcription factors HIF-1 and -2 alpha for ubiquitination under normoxic conditions (12). VHL loss in affected tissues results in HIF stabilization and abnormal expression of hypoxia-inducible genes, such as vascular endothelial growth factor (VEGF). VHL also inhibits transcription elongation (13-16), mRNA stability (17-20), Sp1-related promoter activity (21, 22), and PKC activity (23, 24). Moreover, VHL also promotes induction of morphogenesis, cellular differentiation, contact inhibition (25-29), and fibronectin matrix formation (30). Like Rb, VHL is an unusual tumor suppressor because it inhibits rather than promotes apoptosis, particularly in response to cell stresses, such as serum depletion (31), glucose depletion or endoplasmic reticulum (ER) stress (32), or UV exposure (33). Furthermore, VHL functional heterogeneity is supported by the diversity of cellular compartments where the protein resides, including cytoplasm (34), nucleus (35), mitochondria (36), ER (37), and Golgi apparatus (30). However, VHL functions that are most critical for tumor suppressor activity are as yet unclear, and its biochemical basis is completely unknown.

Genotype-phenotype correlations for VHL protein suggest that additional VHL-mediated tumor suppressor pathways exist. VHL disease retinal angiomas and central nervous system hemangioblastomas are space-occupying, oncogenically benign lesions that result from VHL loss and VEGF overexpression. These lesions correlate precisely with VHL mutations that can be shown biochemically to increase HIF expression (6, 38). In contrast, the overtly malignant renal carcinomas and pancreatic cancers that arise from epithelial cystic lesions through a multi-step process (39) are both pathogenically and genetically distinct from the largely vascular lesions. Moreover, VHL disease 2A mutations, which are not associated with renal cancer (Y98H, Y112H, and G93D), still disrupt IF processing (4-6), indicating that HIF overexpression is insufficient alone to cause renal cancer.

The protein partner of a disease gene product may itself be encoded by a gene for the same disease. Renal cancers arise through a multi-step process (39), which makes elucidation of subsequent genetic events a critical pursuit.

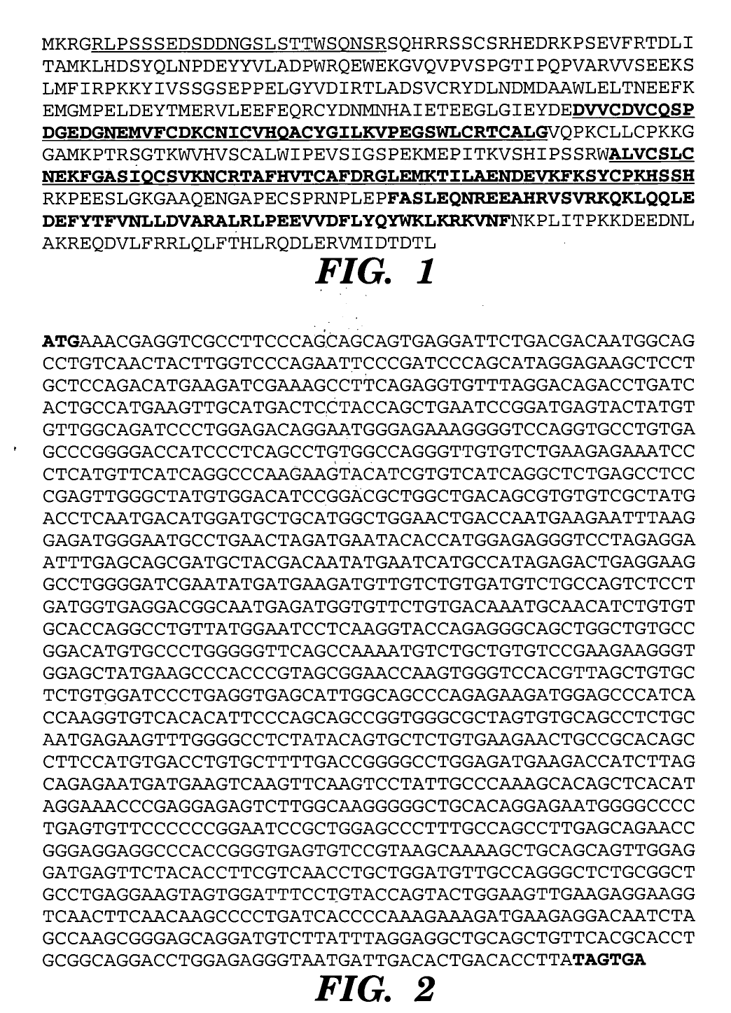

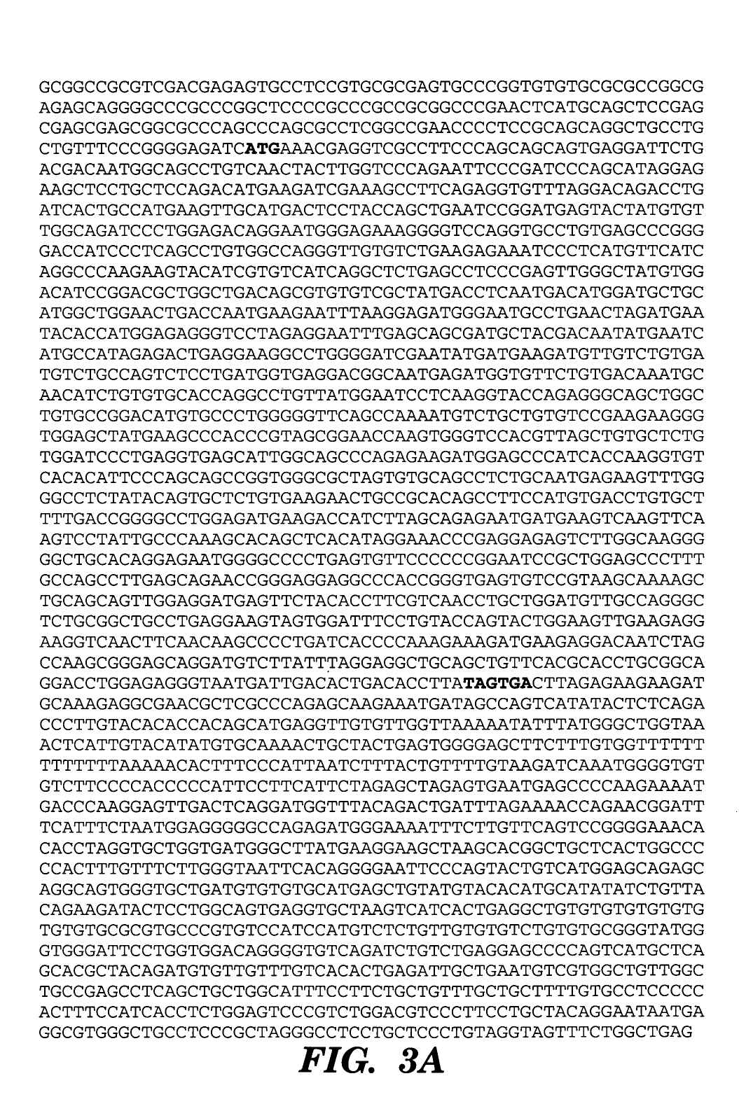

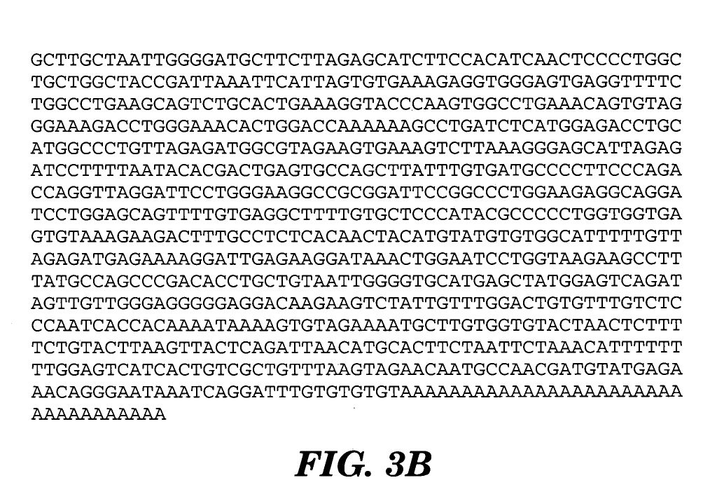

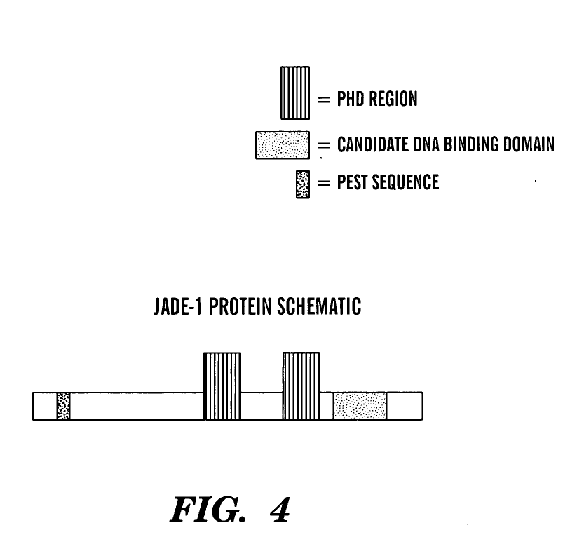

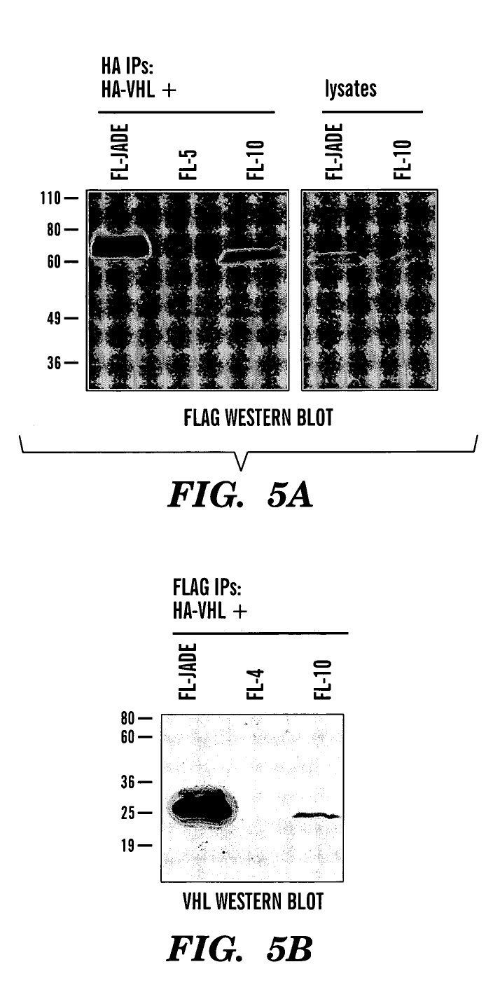

Yeast 2-hybrid approach. Much progress has been made identifying VHL-interacting proteins using immmunoprecipitation approaches. However, VHL is a low abundance protein found in multiple cellular compartments (34-37). To surmount these issues and with an interest in a VHL-mediated renal tumor suppressor pathway, we used the yeast 2-hybrid system to find candidate VHL interactors. Because the relevant tissue is renal proximal tubule, we screened a library from adult human kidney, which is 50% proximal tubule by mass. Fifty of 500,000 clones were positive using full-length human VHL as bait. Twenty-two were chosen for additional studies in mammalian cells. Further description of a novel, full-length clone now called Jade-1 follows (FIGS. 1, 2, 3A-3B and 4). Evidence of the strong interaction on initial coimmunoprecipitations with VHL is shown in FIGS. 5A-5B. Perhaps 30-40% of the Jade-1 in the cell lysate is immunoprecipitable with VHL and vice-versa. This observation suggested that if sufficient Jade-1 and VHL were found in a particular cellular compartment, we would have a high likelihood of demonstrating interaction of the endogenous proteins.

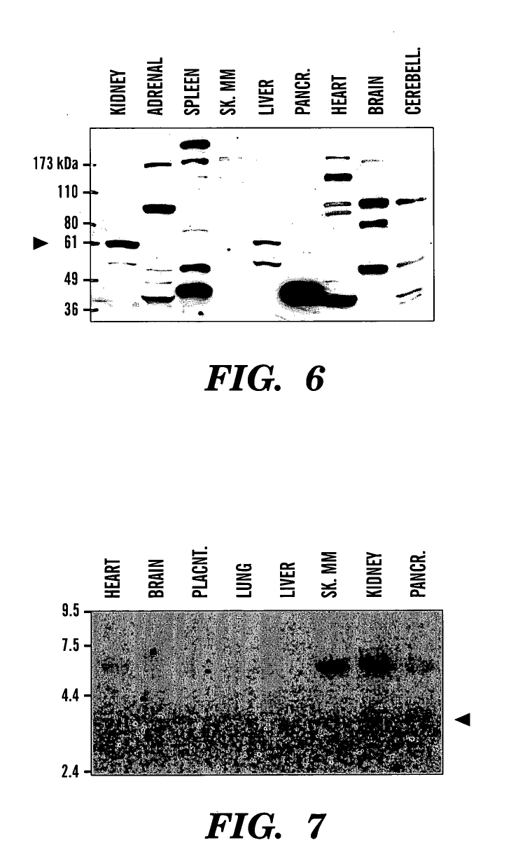

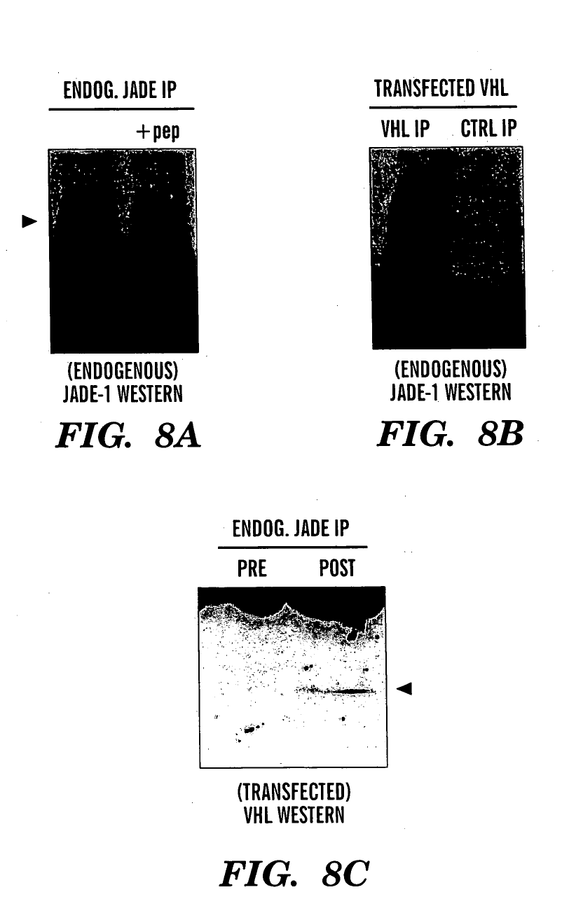

Jade-1 (“gene” for Apoptosis and Differentiation in Epithelial cells) is a 509 amino acid protein that has not been previously characterized, nor have any close homologs. The 5′ end of the transcript was confirmed based on human EST sequences. Jade-1 has 2 PHD or plant homeodomains (8) as its major recognizable features, as well as a divergent homeodomain that might bind DNA, and a candidate PEST sequence (7) as degradation signal near its amino terminus (FIGS. 1, 2, 3A-3B and 4). There are potential O— and N-glycosylation sites and potential S/T and Y-phosphorylation sites. Jade expression appears kidney-enriched (FIGS. 6 and 7). Jade-1 3.6 kb and 6 kb alternatively spliced messages are most prominent in kidney (FIG. 7). An affinity purified antipeptide antiserum directed against the Jade-1 terminal amino acids recognizes a single major band in kidney, and a lesser amount in liver, just larger than the predicted size of 58.4 kDa at about 62 kDa (FIG. 6). Immunoprecipitations of endogenous Jade-1 using this antiserum reveal a prominent major band (FIGS. 8A-8C) (actually a close doublet-see FIG. 15) identical in size to immunoprecipitable untagged, full-length transfected Jade-1 (also a doublet), strongly supporting proper identification of the protein and usefulness of the antiserum. Immunoprecipitation of Jade-1 is completely blocked with the immunizing peptide (FIG. 8A).

Jade-1 alternative splices, homologs, and orthologs: The Jade family. When we first identified the Jade-1 cDNA, there were no corresponding clones, other than EST clones in the GenBank database. Clones identical to the complete Jade-1 509 aa clone, designated XP_{—}033946 and BAB55239 were entered in 2001, although no tissue distribution, protein interaction, or functional information was provided, nor were any publications cited.

The sequences were entered as a result of large-scale efforts to identify cDNA clones. Jade-1 has several alternative splices based on sequences entered into GenBank. Several of these clones are designated as KIAA1807, with 846, 702 (incomplete), 698 and 289 amino acid (aa) versions in GenBank. The 846 aa encoding Jade-1 clone may represent the 6 kb message by Northern blot and would correspond with a 95 kDa protein seen on Western blot (see FIGS. 6 and 7). Based on BLAST database search, a complete Jade-1 genomic clone (Celera genomic clone hCG17335) resides at chromosome band 4q28.1, covers 62.4 kb, and contains 10 Jade-1 p62 exons. Mouse Jade-1 EST clones indicate that there are very close murine homologs of Jade-1 64 kDa, 95 kDa and smaller Jade-1 forms. The mouse Jade-1 64 kDa homolog (61 kDa by Western blotting) nucleotide sequence is incomplete in GenBank, but the amino terminal 20 amino acids are identical between human and mouse 64 (61) kDa Jade-1.

The most homologous Jade family member to Jade-1 is clone KIAA0239 (accession number gi27529704), which is also 79% similar to Jade-1 starting from the first PHD domain onward for 283 aas. There are 286, 498, and 577 aa 850 alternatively spliced versions of clone KIAA0239. In the Celera database, a 766 aa clone from aas 31 to 454 is 65% identical and 75% similar to Jade-1 aas 51 to 502. The next most similar family member is clone KIAA0215, the E9 polypeptide, which is an 823 aa protein that is 59% identical and 74% similar to Jade-1 aas 1-502. Sequence analysis of this Drosophila clone beyond the Jade-1 homology region reveals almost no homologies by BLAST search, but does identify a high-scoring WSC B domain at aas 783 to 829 (E-value 0.00064) by BLOCK search. WSC proteins contain 3 characteristic domains (A, B, and C) that are thought to bind carbohydrate and, interestingly, all of which are found in the PKD1 family of proteins. WSC proteins are transmembrane proteins involved in maintaining cell wall integrity and contributing to stress response (heat shock) in yeast (Verna-J et al PNAS 97). Moreover, yeast WSC proteins present in the secretory path are required for arrest of the secretory response (ASR), suggesting that accumulated WSC proteins initiate the ASR (Nanduri-J Mol Cell 2001). WSC2p is the family member required for the ASR. This ASR pathway is also distinct from ER stress-related unfolded protein response. The effect of the WSC family in ASR may be mediated by PKC1-MPK1 (Verna, Nanduri). C. elegans 744 aa AAF59546 protein from aas 138 to 508 is 46% similar to Jade-1 over aas 71 to 458. Little additional homology is identifiable beyond the Jade-1 related region by BLOCKs or BLAST searches. Drosophila clone CG1845 is a 1430 aa weaker homolog (47% similarity) over Jade-1 aas 136 to 501 aas. CG1845 contains a bromodomain (aas 646 to 691), a strong probable PWWP A domain (aas 1309 to 1323) and also a possible WSC C domain (aas 930 to 951) with BLOCKs E-value of 0.056. Other Jade-1 homologs contain bromodomains. For example the 1214 aa protein Br140 or Peregrin, which maps to chromosome 3p25, over aas 210 to 600 is 44% similar to Jade-1 aas 135 to 501.

Other related bromodomain-containing proteins include BRL and BRF3 (KIAA1286), which appears to be related or the same as a different Br140 splice. Several of the genes involved in acute myelogenous leukemia, such as MLL (human homologue of Drosophile trithorax), AF10, AF17 have high homology to the Jade-1 PHD region, including PHD1 PHD2 and the interPHD region. The latter with PHD2 has also been given a designation of extended PHD or extended LAP domain. Similar PHD/extended PHD features are found in human clones KIAA0783, KIAA0780, KIAA0876 and KIAA0677, Drosophila Alhambra and trithorax, and C. elegans LIN-49, CEZF, Saccharomyces cerevisiae Nto1p and Schizosaccharomyces pombe T39715. All of these proteins can be considered as members of the Jade family. Other members of the family may be subsequently identified. We expect that members of the Jade family will have at least 20% amino acid similarity over a conserved Jade protein domain, such as a single Jade PHD region, for example. Thus, the listing of Jade family members above is partial, not complete.

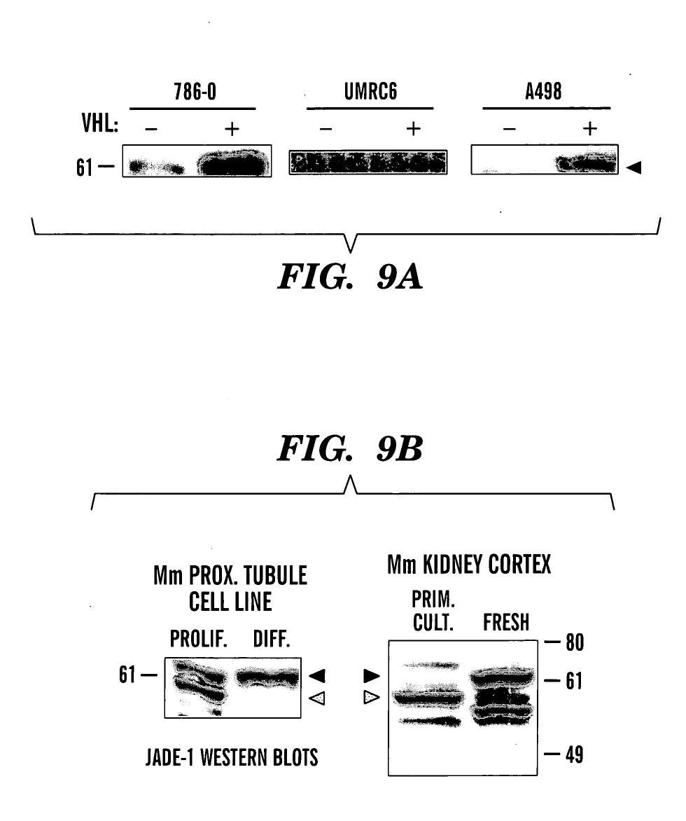

Jade-1 is upregulated by VHL and differentiation of proximal tubule cells. As shown in FIG. 9A, endogenous Jade-1 protein is upregulated in 3 of 3 different renal cancer cell lines with stable reintroduction of wild-type VHL, which has been confirmed as well by Jade-1 immunoprecipitation (data not shown). Even UMRC6 cells with wild-type VHL detectable only by Northern blot exhibit increased Jade-1 expression. FIG. 9B shows Jade-1 protein expressed in the tissue of interest, in a mouse renal proximal tubule cell line, and can be upregulated with differentiation of the cells. In addition, a similar phenomenon is observed in fresh mouse renal cortex (80% proximal tubules by mass), which has abundant Jade-1 that is strongly downregulated with primary culture.

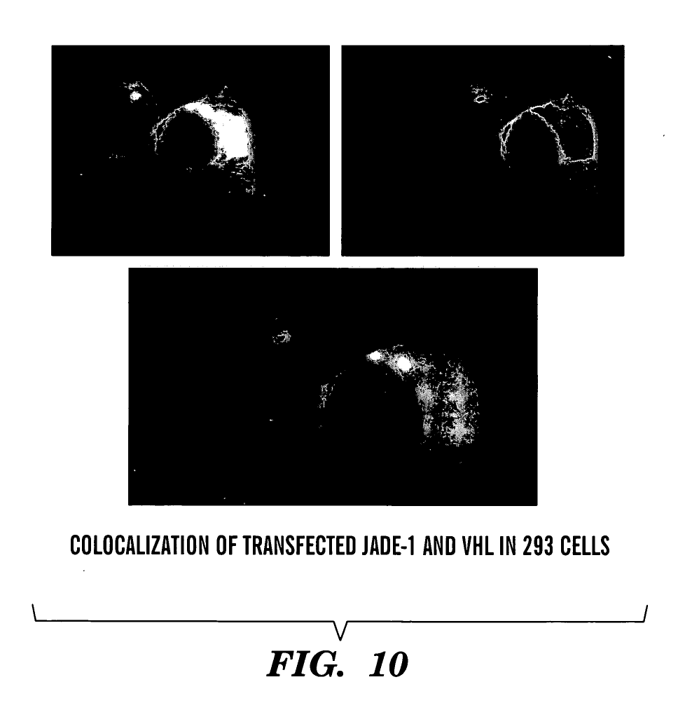

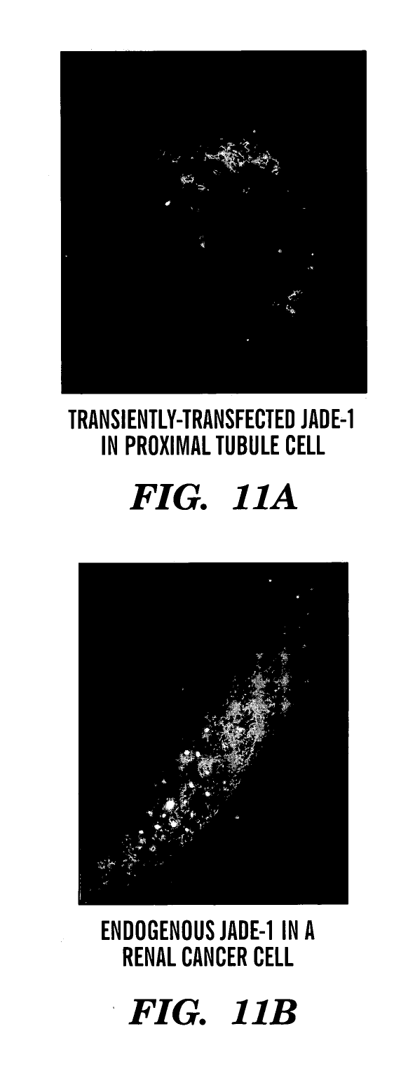

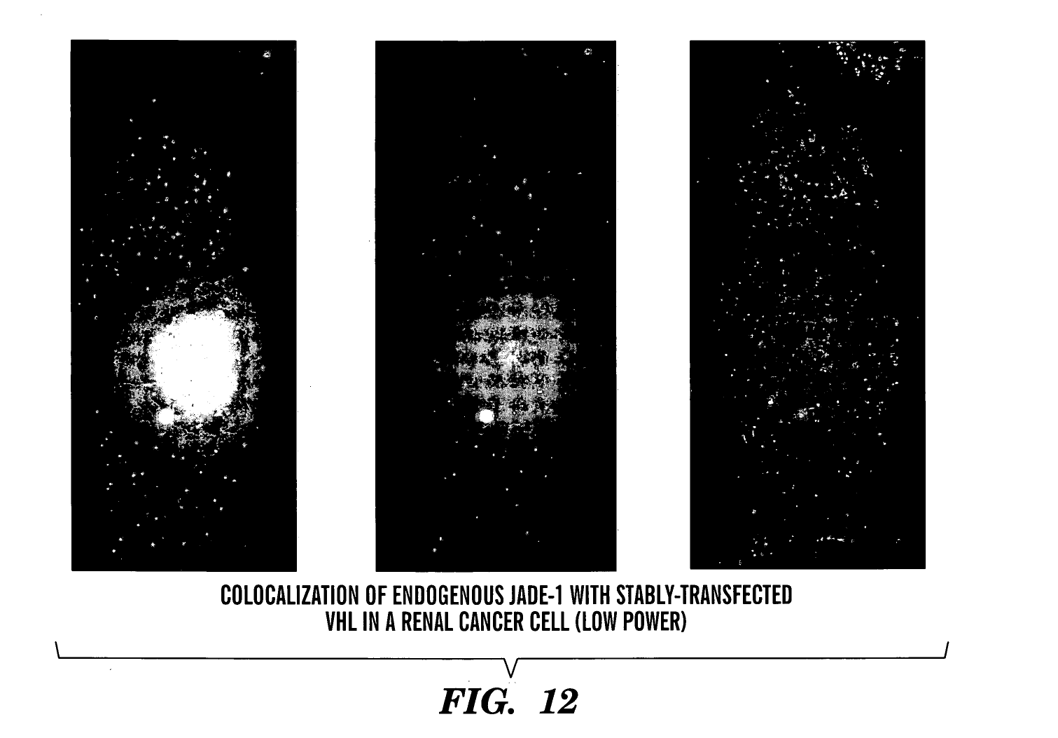

Binding and colocalization of Jade-1 and VHL. FIG. 8 shows that endogenous Jade-1 binds transiently transfected VHL in 293 cells. Similar observations have been made using stably transfected renal cancer cells (data not shown). FIG. 10 shows complete colocalization of transiently transfected Jade-1 and VHL in 293 cells. There is no signal bleed-through to either channel. FIG. 11A demonstates transfected, tagged Jade-1 localization to cytoplasm and nuclear speckles using a mono-specific anti-tag antibody. Prominent nuclear speckles are also observed using the affinity purified anti-Jade-1 serum in renal cancer cells, as well as diffuse cytoplasmic staining and cytoplasmic speckles (FIG. 11B). The nuclear speckles suggest Jade-1 may play a role in regulating gene expression, as do other PHD proteins. Colocalization of endogenous Jade-1 and stably transfected VHL is shown in FIG. 12, in which there is diffuse nuclear VHL and Jade-1 in this cell, as well as colocalization to cytoplasmic speckles in the same cell (FIG. 13). VHL in cytoplasmic speckles colocalizes with endoplasmic reticulum markers (37).

Jade-1: A role in stress-induced apoptosis. The first clue to Jade-1 function arose from attempts to stably overexpress it in 786-O renal cancer cells. We typically achieve 30% success rates of drug-resistant 786-O colonies expressing a transgene, and even 10% success with VHL, which is a growth suppressor. However none of the Jade-1-transfected, drug-resistant colonies expressed exogenous Jade-1, suggesting it might also be growth suppressive. We therefore tested whether transient transfection of 293 cells with a Jade-1 expression vector might cause apoptosis. We used Hoechst 33342 as a specific indicator of apoptosis and found that Jade-1 transfection doubles the level of apoptosis observed with empty vector transfected cells (FIG. 14, left). VHL transfection also increases apoptosis. Intriguingly, cotransfection of Jade-1 and VHL reduces overall apoptosis, while an additive increase in apoptosis was expected. Not shown, VHL truncations and missense mutations do cause additive increases in apoptosis. These observations raise the possibility that VHL, as an inhibitor of stress-induced apoptosis (31-33), might prevent apoptosis mediated by Jade-1. In keeping with this hypothesis, an antisense Jade-1 expression vector is able to block the proapoptotic effect of serum depletion (FIG. 14, right) or UV-induced apoptosis (data not shown). Western analysis shows that Jade-1 antisense substantially reduces Jade-1 expression levels, and in response to serum depletion, also decreases caspase-3-mediated poly-(ADP-ribose)-polymerase (PARP) cleavage and increases levels of Bcl2 (FIG. 15), which is antiapoptotic. These results indicate that Jade-1 antisense is protective. Because VHL acts to inhibit stress-induced apoptosis in part by increasing Bcl2 levels (69), Jade-1 may therefore play an active apoptotic role in a VHL pathway.

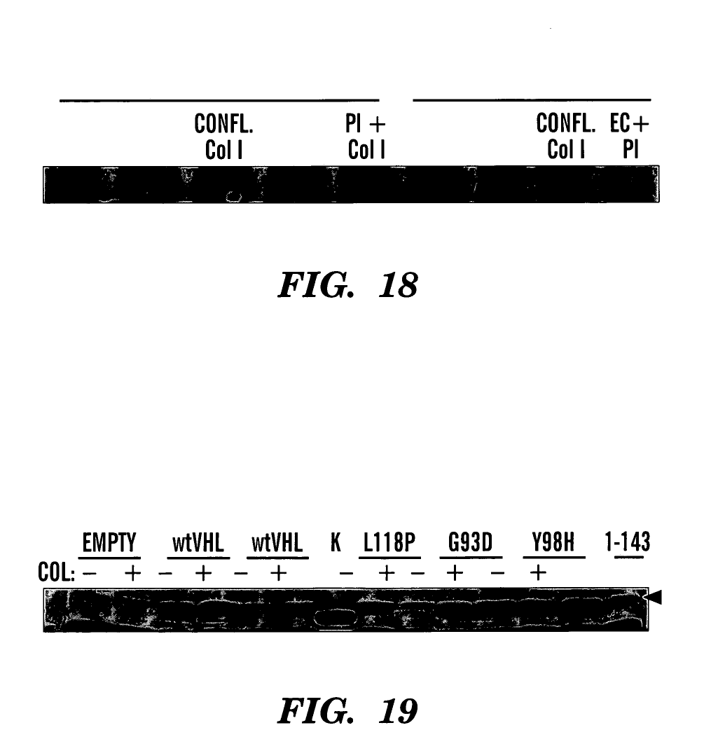

VHL increases both endogenous and transfected Jade-1 protein abundance. Because wild-type VHL increases endogenous Jade-1 protein abundance in renal cancer cells (FIG. 9A), we tested the effect of transiently contransfected VHL on Jade-1. Interestingly, we saw no difference in Jade-1 expression with or without VHL 1 day after transfection (FIG. 16). However, 3 days after transfection VHL cotransfected cells express much more Jade-1 than empty vector or beta galactosidase cotransfected cells. An even greater expression differential is seen with HeLa or fibrosarcoma cells (FIG. 16). Preliminary metabolic labeling experiments suggest that VHL increases Jade-1 protein half-life (FIG. 17). Interestingly, although Jade-1 binds many VHL truncations and missense mutations as well as wild-type VHL, when tested 1 day post-transfection (data not shown), preliminary experiments indicate that only wild-type or mild VHL missense mutations maximally increase Jade-1 abundance at 3 days. These experiments make an intriguing distinction between binding and stabilization, supporting a biologic consequence of the protein-protein interaction and a possible disease relationship between the proteins. Similarly, endogenous Jade-1 protein is increased with proteasome inhibition in VHL minus renal cancer cells and less so in VHL plus renal cancer cells (FIG. 18), suggesting again that Jade-1 turnover is rapid, and also supporting the cotransfection data that VHL protects Jade-1 from proteasomal degradation, which represents a novel VHL function. Most importantly, endogenous Jade-1 is increased in 786-O renal cancer cells stably expressing wild-type VHL or VHL missense mutations that do not cause renal cancer, whereas renal cancer-causing VHL mutations downregulate Jade-1 when cells are grown to high confluence on collagen (FIG. 19). Therefore, transiently transfected Jade-1 and VHL likely model the endogenous proteins. Moreover, Jade-1 is a strong candidate to participate in VHL-mediated renal tumor suppression. Jade-1 expression is increased and the protein half-life is prolonged using proteasome inhibitors or calpain inhibitors.

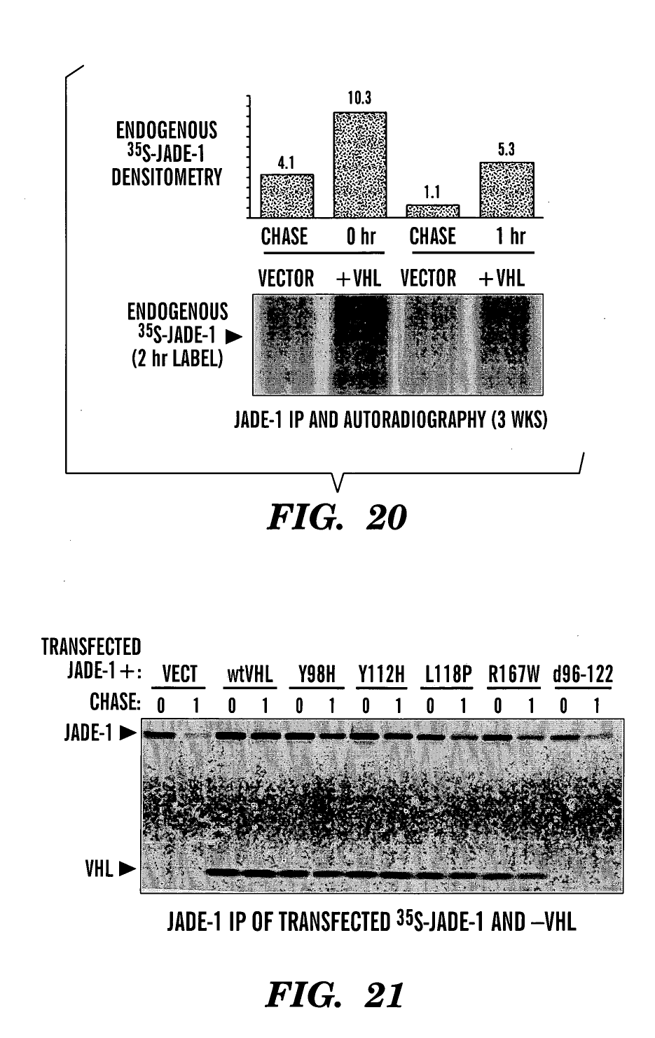

Endogenous Jade-1 is stabilized by stably-transfected VHL in renal cancer cells. We performed metabolic labeling of endogenous Jade-1 and confirmed the effect of VHL on endogenous Jade-1 stability (FIG. 20). In VHL-minus 786-O renal cancer cells, the endogenous Jade-1 protein half-life is ˜40 minutes, whereas in VHL-plus 786-O renal cancer cells, the endogenous Jade-1 protein half-life is ˜60 minutes (FIG. 20). This increase in endogenous Jade-1 stability is quite similar to the increase in stability Jade-1 transiently transfected with VHL, in which Jade-1 half-life increases from 40 minutes to 90 minutes (data not shown). This set of observations further supports the validity of the VHL-Jade-1 interaction and the reliability of the transient transfection system as a model for the endogenous proteins.

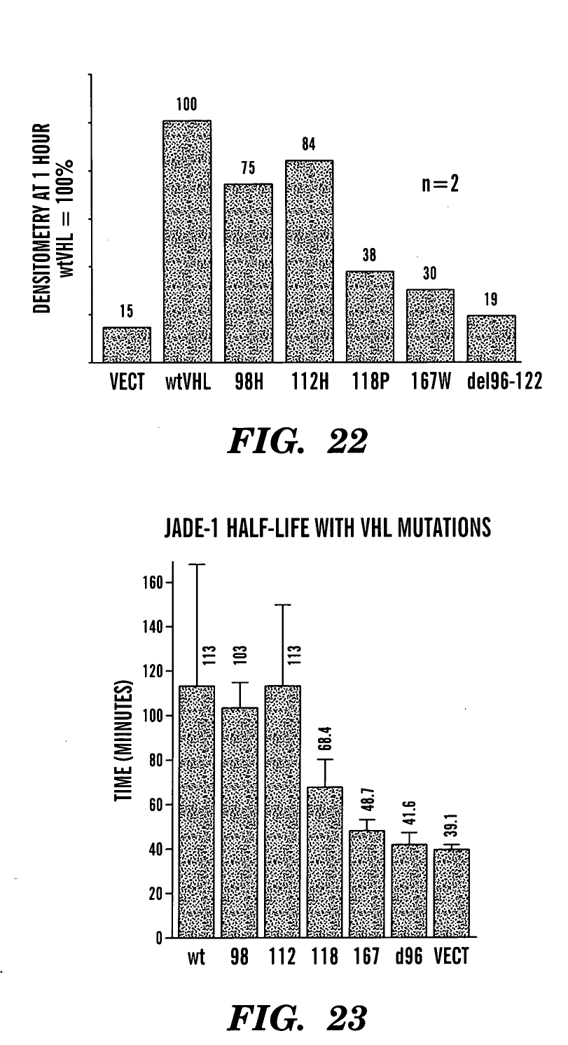

VHL missense mutations that do not cause renal cancer can stabilize Jade-1, but renal cancer causing mutations stabilize Jade-1 only modestly. We have also shown in cotransfection experiments using metabolic labeling that non-renal cancer causing wild-type and missense VHL mutations increase Jade-1 half life, whereas renal cancer-causing VHL mutations do not increase Jade-1 half life to the same extent (FIGS. 21 and 22). Additional experiments will be performed and results will be combined to demonstrate statistical significance by ANOVA. We are testing other VHL missense mutations as well. These observations support the findings from FIG. 19 of different VHL mutations on endogenous Jade-1 expression. We have observed similar findings with VHL truncations. VHL 1-115, 1-143, and 1-175 are all unable to stabilize Jade-1. Together, these findings convincingly make the point that mutations or deletions in either the VHL alpha domain (amino acids 157-190) or beta domain (amino acids 1-156) can disrupt VHL stabilization of Jade-1, suggesting that the entire VHL protein is required. Moreover, the biochemical relationship between VHL mutations and Jade-1 expression fits better with the development of renal cancer than any VHL protein-protein interaction described thus far.

We have also done some additional control experiments that demonstrate the specificity of VHL for stabilization of Jade-1 over other proteins. We have done cotransfection experiments in 293 cells with VHL and Sp1 and PKC-zeta, which are other VHL-interacting proteins. We have also tested p53 with VHL. Convincingly, VHL is unable to stabilize expression of Sp1, PKC-zeta, or p53, supporting the specificity of the Jade-1 stabilization (data not shown). In summary, these observations support the validity of the VHL-Jade-1 interaction and the clinical relevance of the pathway being explored.

Jade-1 is highly regulated. In addition to upregulation with differentiation and downregulation with dedifferentiation of renal epithelial cells and according to VHL status, Jade-1 is regulated by a variety of cellular stimuli. We have observed downregulation of Jade-1 protein with serum deprivation, upregulation with proteasome inhibitors (FIG. 18) and with hypoxia (FIG. 23). These observations support the idea that Jade-1 protein is highly regulated by physiologically relevant stimuli and by agents that can be administered therapeutically. In addition, these observations suggest that Jade-1 may participate in pro- or anti-apoptotic events in response to these stimuli, or in other processes in response or conjunction with these stimuli.

References

1. Gnarra J R, Tory K, Weng Y, Schmidt L, Wei M H, Li H, et al. Mutations of the VHL tumour suppressor gene in renal carcinoma. Nat Genet 1994; 7(1):85-90.
2. Herman J G, Latif F, Weng Y, Lerman M I, Zbar B, Liu S, et al. Silencing of the VHL tumor-suppressor gene by DNA methylation in renal carcinoma Proc Natl Acad Sci USA 1994;91(21):9700-4.
3. Ivan M, Kaelin W G, Jr. The von Hippel-Lindau tumor suppressor protein. Curr Opin Genet Dev 2001;11(1):27-34.
4. Cockman M E, Masson N, Mole D R, Jaakkola P, Chang G W, Clifford S C, et al. Hypoxia inducible factor-alpha binding and ubiquitylation by the von Hippel-Lindau tumor suppressor protein. J Biol Chem 2000;275(33):25733-41.
5. Ohh M, Park C W, Ivan M, Hoffman M A, Kim T Y Huang L E, et al. Ubiquitination of hypoxia-inducible factor requires direct binding to the beta-domain of the von Hippel-Lindau protein. Nat Cell Biol 2000;2(7):423-7.
6. Clifford S C, Cockman M E, Smallwood A C, Mole D R, Woodward E R, Maxwell P H, et al. Contrasting effects on HIF-alpha regulation by disease-causing pVHL mutations correlate with patterns of tumourigenesis in von Hippel-Lindau disease. Hum Mol Genet 2001;10(10):1029-38.
7. Rechsteiner M, Rogers S W. PEST sequences and regulation by proteolysis. Trends Biochem Sci 996;21(7):267-71.
8. Aasland R, Gibson T J, Stewart A F. The PHD finger: implications for chromatin-mediated transcriptional regulation. Trends Biochem Sci 1995;20(2):56-9.
9. Saha V, Chaplin T, Gregorini A, Ayton P, Young B D. The leukemia-associated-protein (LAP) domain, a cysteine-rich motif, is present in a wide range of proteins, including MLL, AF10, and MLLT6 proteins. Proc Natl Acad Sci USA 1995;92(21):9737-41.
10. Kugler A, Stuhler G, Walden P, Zoller G, Zobywalski A, Brossart P, et al. Regression of human metastatic renal cell carcinoma after vaccination with tumor cell-dendritic cell hybrids. Nat Med 2000;6(3):332-6.
11. Latif F, Tory K, Gnarra J, Yao M, Duh F M, Orcutt M L, et al. Identification of the von Hippel-Lindau disease tumor suppressor gene (see comments). Science 1993;260(5112):1317-20.
12. Maxwell P H, Wiesener M S, Chang G W, Clifford S C, Vaux E C, Cockman M E, et al. The tumour suppressor protein VHL targets hypoxia-inducible factors for oxygen-dependent proteolysis. Nature 1999;399(6733):271-5.
13. Aso T, Lane W S, Conaway J W, Conaway R C. Elongin (SIII): a multisubunit regulator of elongation by RNA polymerase II (see comments). Science 1995;269(5229):1439-43.
14. Duan D R, Pause A, Burgess W H, Aso T, Chen D Y, Garrett K P, et al. Inhibition of transcription elongation by the VHL tumor suppressor protein (see comments). Science 1995;269(5229):1402-6.
15. Kibel A, Iliopoulos O, DeCaprio J A, Kaelin W G, Jr. Binding of the von Hippel-Lindau tumor suppressor protein to Elongin B and C (see comments). Science 1995;269(5229):1444-6.
16. Kroll S L, Paulding W R, Schnell P O, Barton M C, Conaway J W, Conaway R C, et al. von Hippel-Lindau protein induces hypoxia-regulated arrest of tyrosine hydroxylase transcript elongation in pheochromocytoma cells. J Biol Chem 1999;274(42):30109-14.
17. Iliopoulos O, Levy A P, Jiang C, Kaelin W G, Jr., Goldberg M A. Negative regulation of hypoxia-inducible genes by the von Hippel-Lindau protein. Proc Nat Acad Sci USA 1996;93(20):10595-9.
18. Gnarra J R, Zhou S, Merrill M J, Wagner J R, Krumm A, Papavassiliou E, et al. Post-transcriptional regulation of vascular endothelial growth factor mRNA by the product of the VHL tumor suppressor gene. Proc Natl Acad Sci USA 1996;93(20):10589-94.
19. Levy A P, Levy N S, Goldberg M A. Hypoxia-inducible protein binding to vascular endothelial growth factor mRNA and its modulation by the von Hippel-Lindau protein. J Biol Chem 1996;271(41):25492-7.
20. Pioli P A, Rigby W F. The von hippel-lindau protein interacts with heteronuclear ribonucleoprotein a2 and regulates its expression. J Biol Chem 2001;276(43):40346-52.
21. Mukhopadhyay D, Knebelmann B, Cohen H T, Ananth S, Sukhatme V P. The von Hippel-Lindau tumor suppressor gene product interacts with Sp1 to repress vascular endothelial growth factor promoter activity. Mol Cell Biol 1997;17(9):5629-39.
22. Cohen H T, Zhou M, Welsh A M, Zarghamee S, Scholz H, Mukhopadhyay D, et al. An important von Hippel-Lindau tumor suppressor domain mediates Sp1-binding and self-association. Biochem Biophys Res Commun 1999;266(1):43-50.
23. Pal S, Claffey K P, Dvorak H F, Mukhopadhyay D. The von Hippel-Lindau gene product inhibits vascular permeability factor/vascular endothelial growth factor expression in renal cell carcinoma by blocking protein kinase C pathways. J Biol Chem 1997;272(44):27509-12.
24. Pal S, Claffey K P, Cohen H T, Mukhopadhyay D. Activation of Sp1-mediated vascular permeability factor/vascular endothelial growth factor transcription requires specific interaction with protein kinase C zeta. J Biol Chem 1998;273(41):26277-80.
25. Koochekpour S, Jeffers M, Wang P H, Gong C, Taylor G A, Roessler L M, et al. The von Hippel-Lindau tumor suppressor gene inhibits hepatocyte growth factor/scatter factor-induced invasion and branching morphogenesis in renal carcinoma cells. Mol Cell Biol 1999;19(9):5902-12.
26. Lieubeau-Teillet B, Rak J, Jothy S, Iliopoulos O, Kaelin W, Kerbel R S. von Hippel-Lindau gene-mediated growth suppression and induction of differentiation in renal cell carcinoma cells grown as multicellular tumor spheroids. Cancer Res 1998;58(21):4957-62.
27. Davidowitz E J, Schoenfeld A R, Burk R D. VHL induces renal cell differentiation and growth arrest through integration of cell-cell and cell-extracellular matrix signaling. Mol Cell Biol 2001;21(3):865-74.
28. Kamada M, Suzuki K, Kato Y, Okuda H, Shuin T. von Hippel-Lindau protein promotes the assembly of actin and vinculin and inhibits cell motility. Cancer Res 2001;61(10):4184-9.
29. Baba M, Hirai S, Kawakami S, Kishida T, Sakai N, Kaneko S, et al. Tumor suppressor protein VHL is induced at high cell density and mediates contact inhibition of cell growth. Oncogene 2001;20(22):2727-36.
30. Ohh M, Yauch R L, Lonergan K M, Whaley J M, Stemmer-Rachamimov A O, Louis D N, et al. The von Hippel-Lindau tumor suppressor protein is required for proper assembly of an extracellular fibronectin matrix. Mol Cell 1998;1(7):959-68.
31. Pause A, Lee S, Lonergan K M, Klausner R D. The von Hippel-Lindau tumor suppressor gene is required for cell cycle exit upon serum withdrawal. Proc Natl Acad Sci USA 1998;95(3):993-8.
32. Gorospe M, Egan J M, Zbar B, Lerman M, Geil L, Kuzmin I, et al. Protective function of von Hippel-Lindau protein against impaired protein processing in renal carcinoma cells. Mol Cell Biol 1999;19(2):1289-300.
33. Schoenfeld A R, Parris T, Eisenberger A, Davidowitz E J, De Leon M, Talasazan F, et al. The von Hippel-Lindau tumor suppressor gene protects cells from UV-mediated apoptosis. Oncogene 2000;19(51):5851-7.
34. Los M, Jansen G H, Kaelin W G, Lips C J, Blijham G H, Voest E E. Expression pattern of the von Hippel-Lindau protein in human tissues. Lab Invest 1996;75(2):231-8.
35. Lee S, Chen D Y, Humphrey J S, Gnarra J R, Linehan W M, Klausner R D. Nuclear/cytoplasmic localization of the von Hippel-Lindau tumor suppressor gene product is determined by cell density. Proc Natl Acad Sci USA 1996;93(5):1770-5.
36. Shiao Y H, Resau J H, Nagashima K, Anderson L M, Ramakrishna G. The von Hippel-Lindau tumor suppressor targets to mitochondria. Cancer Res 2000;60(11):2816-9.
37. Schoenfeld A R, Davidowitz E J, Burk R D. Endoplasmic reticulum/cytosolic localization of von Hippel-Lindau gene products is mediated by a 64-amino acid region. Int J Cancer 2001;91(4):457-67.
38. Hoffman M A, Ohh M, Yang H, Klco J M, Ivan M, Kaelin W G, Jr. von Hippel-Lindau protein mutants linked to type 2C VHL disease preserve the ability to downregulate HIF. Hum Mol Genet 2001;10(10):1019-27.
39. Lubensky I A, Gnarra J R, Bertheau P, Walther M M, Linehan W M, Zhuang Z. Allelic deletions of the VHL gene detected in multiple microscopic clear cell renal lesions in von Hippel-Lindau disease patients. Am J Pathol 1996;149(6):2089-94.
40. Jiang F, Desper R, Papadimitriou C H, Schaffer A A, Kallioniemi O P, Richter J, et al. Construction of evolutionary tree models for renal cell carcinoma from comparative genomic hybridization data. Cancer Res 2000;60(22):6503-9.
41. Yang Z Q, Yoshida M A, Fukuda Y, Kurihara N, Nakamura Y, Inazawa J. Molecular cytogenetic analysis of 17 renal cancer cell lines: increased copy number at 5q31-33 in cell lines from nonpapillary carcinomas. Jpn J Cancer Res 2000;91(2):156-63.
42. Schraml P, Zhaou M, Richter J, Bruning T, Pommer M, Sauter G, et al. (Analysis of kidney tumors in trichloroethylene exposed workers by comparative genomic hybridization and DNA sequence analysis). Verh Dtsch Ges Pathol 1999;83:218-24.
43. Jiang F, Richter J, Schraml P, Bubendorf L, Gasser T, Sauter G, et al. Chromosomal imbalances in papillary renal cell carcinoma: genetic differences between histological subtypes. Am J Pathol 1998;153(5):1467-73.
44. Klamt B, Schulze M, Thate C, Mares J, Goetz P, Kodet R, et al. Allele loss in Wilms tumors of chromosome arms 11q, 16q, and 22q correlate with clinicopatholgical parameters. Genes Chromosomes Cancer 1998;22(4):287-94.
45. Steenman M, Redeker B, de Meulemeester M, Wiesmeiyer K, Voute P A, Westerveld A, et al. Comparative genomic hybridization analysis of Wilms tumors. Cytogenet Cell Genet 1997;77(3-4):296-303.
46. Jiang F, Moch H, Richter J, Egenter C, Gasser T, Bubendorf L, et al. Comparative genomic hybridization reveals frequent chromosome 13q and 4q losses in renal carcinomas with sarcomatoid transformation. J Pathol 1998;185(4):382-8.
47. Gibbons R J, Bachoo S, Picketts D J, Aftimos S, Asenbauer B, Bergoffen J, et al. Mutations in transcriptional regulator ATRX establish the functional significance of a PHD-like domain. Nat Genet 1997;17(2):146-8.
48. An autoimmune disease, APECED, caused by mutations in a novel gene featuring two PHD-type zinc-finger domains. The Finnish-German APECED Consortium. Autoimmune Polyendocrinopathy-Candidiasis-Ectodermal Dystrophy. Nat Genet 1997;17(4):399-403.
49. Nagamine K, Peterson P, Scott H S, Kudoh J, Minoshima S, Heino M, et al. Positional cloning of the APECED gene. Nat Genet 1997;17(4):393-8.
50. Gunduz M, Ouchida M, Fukushima K, Hanafusa H, Etani T, Nishioka S, et al. Genomic structure of the human ING1 gene and tumor-specific mutations detected in head and neck squamous cell carcinomas. Cancer Res 2000;60(12):3143-6.
51. Chen L, Matsubara N, Yoshino T, Nagasaka T, Hoshizima N, Shirakawa Y, et al. Genetic alterations of candidate tumor suppressor ING1 in human esophageal squamous cell cancer. Cancer Res 2001;61(11):4345-9.
52. Hayashi Y. The molecular genetics of recurring chromosome abnormalities in acute myeloid leukemia. Semin Hematol 2000;37(4):368-80.
53. Bjorses P, Halonen M, Palvimo J J, Kolmer M, Aaltonen J, Ellonen P, et al. Mutations in the AIRE gene: effects on subcellular location and transactivation function of the autoimmune polyendocrinopathy-candidiasis-ectodermal dystrophy protein. Am J Hum Genet 2000; 66(2):378-92.
54. Pitkanen J, Doucas V, Sternsdorf T, Nakajima T, Aratani S, Jensen K, et al. The autoimmune regulator protein has transcriptional transactivating properties and interacts with the common coactivator CREB-binding protein. J Biol Chem 2000;275(22):16802-9.
55. Schultz D C, Friedman J R, Rauscher F J, 3rd. Targeting histone deacetylase complexes via KRAB-zinc finger proteins: the PHD and bromodomains of KAP-1 form a cooperative unit that recruits a novel isoform of the Mi-2alpha subunit of NuRD. Genes Dev 2001;15(4):428-43.
56. Schindler U, Beckmann H, Cashmore A R. HAT3.1, a novel Arabidopsis homeodomain protein containing a conserved cysteine-rich region. Plant J 1993;4(1):137-50.
57. McDowell T L, Gibbons R J, Sutherland H, O'Rourke D M, Bickmore W A, Pombo A, et al. Localization of a putative transcriptional regulator (ATRX) at pericentromeric heterochromatin and the short arms of acrocentric chromosomes. Proc Natl Acad Sci USA 1999;96(24):13983-8.
58. Bjorses P, Pelto-Huikko M, Kaukonen J, Aaltonen J, Peltonen L, Ulmanen I. Localization of the APECED protein in distinct nuclear structures. Hum Mol Genet 1999;8(2):259-66.
59. Nakamura T, Blechman J, Tada S, Rozovskaia T, Itoyama T, Bullrich F, et al. huASH1 protein, a putative transcription factor encoded by a human homologue of the Drosophila ash1 gene, localizes to both nuclei and cell-cell tight junctions. Proc Natl Acad Sci USA 2000;97(13):7284-9.
60. Gabig T G, Mantel P L, Rosli R, Crean C D. Requiem: a novel zinc finger gene essential for apoptosis in myeloid cells. J Biol Chem 1994;269(47):29515-9.
61. Helbing C C, Veillette C, Riabowol K, Johnston R N, Garkavtsev I. A novel candidate tumor suppressor, ING1, is involved in the regulation of apoptosis. Cancer Res 1997;57(7):1255-8.
62. Garkavtsev I, Kazarov A, Gudkov A, Riabowol K. Suppression of the novel growth inhibitor p33ING1 promotes neoplastic transformation. Nat Genet 1996;14(4):415-20.
63. Garkavtsev I, Grigorian I A, Ossovskaya V S, Chernov M V, Chumakov P M, Gudkov A V. The candidate tumour suppressor p33ING1 cooperates with p53 in cell growth control Nature 1998;391(6664):295-8.
64. Garkavtsev I, Riabowol K. Extension of the replicative life span of human diploid fibroblasts by inhibition of the p33ING1 candidate tumor suppressor. Mol Cell Biol 1997;17(4):2014-9.
65. Siemeister G, Weindel K, Mohrs K, Barleon B, Martiny-Baron G, Marme D. Reversion of deregulated expression of vascular endothelial growth factor in human renal carcinoma cells by von Hippel-Lindau tumor suppressor protein. Cancer Res 1996;56(10):2299-301.
66. Zhou M, Wang H, Ross J J, Mahmud H A, Xu C, Cohen H T. A novel protein that interacts with the von Hippel-Lindau gene product resides in cytoplasmic and nuclear speckles in proximal tubule cells. J Am Soc Nephrol 2001;12:625P.
67. Arnould T, Sellin L, Benzing T, Tsiokas L, Cohen H T, Kim E, et al. Cellular activation triggered by the autosomal dominant polycystic kidney disease gene product PKD2. Mol Cell Biol 1999;19(5):3423-34.
68. Datta K, Nambudripad R, Pal S, Zhou M, Cohen H T, Mukhopadhyay D. Inhibition of insulin-like growth factor-I-mediated cell signaling by the von Hippel-Lindau gene product in renal cancer. J Biol Chem 2000;275(27):20700-6.
69. Devarajan P, De Leon M, Talasazan F, Schoenfeld A R, Davidowitz E J, Burk R D. The von Hippel-Lindau gene product inhibits renal cell apoptosis via Bcl-2-dependent pathways. J Biol Chem 2001;20:20.
70. Okuda H, Saitoh K, Hirai Si S, Iwai K, Takaki Y, Baba M, et al. The Von Hippel-Lindau (VHL) tumor suppressor protein mediates ubiquitination of activated a typical protein kinase C J Biol Chem 2001;26:26.
71. Kim M, Katayose Y, Li Q, Rakkar A N, Li Z, Hwang S G, et al. Recombinant adenovirus expressing Von Hippel-Lindau-mediated cell cycle arrest is associated with the induction of cyclin-dependent kinase inhibitor p27Kip1. Biochem Biophys Res Commun 1998;253(3):672-7.
72. Liakopoulos D, Busgen T, Brychzy A, Jentsch S, Pause A. Conjugation of the ubiquitin-like protein NEDD8 to cullin-2 is linked to von Hippel-Lindau tumor suppressor function. Proc Natl Acad Sci USA 1999;96(10):5510-5.
73. Wada H, Yeh E T, Kamitani T. The von Hippel-Lindau tumor suppressor gene product promotes, but is not essential for, NEDD8 conjugation to cullin-2. J Biol Chem 1999;274(50):36025-9.
74. Lisztwan J, Imbert G, Wirbelauer C, Gstaiger M, Krek W. The von Hippel-Lindau tumor suppressor protein is a component of an E3 ubiquitin-protein ligase activity. Genes Dev 1999;13(14):1822-33.
75. Haase V H, Glickman J N, Socolovsky M, Jaenisch R. Vascular tumors in livers with targeted inactivation of the von Hippel-Lindau tumor suppressor. Proc Natl Acad Sci USA 2001;98(4):1583-8.
76. Dierick A M, Praet M, Roels H, Verbeeck P, Robyns C, Oosterlinck W. Vimentin expression of renal cell carcinoma in relation to DNA content and histological grading: a combined light microscopic, immunocytochemical and cytophotometrical analysis. Histopathology 1991;18(4):315-22.
77. Chang K S, Fan Y H, Andreeff M, Liu J, Mu Z M. The PML gene encodes a phosphoprotein associated with the nuclear matrix. Blood 1995;85(12):3646-53.
78. Cohen H T, Bossone S A, Zhu G, McDonald G A, Sukhatme V P. Sp1 is a critical regulator of the Wilms' tumor-1 gene. J Biol Chem 1997;272(5):2901-13.
79. Shumway S D, Maki M, Miyamoto S. The PEST domain of IkappaBalpha is necessary and sufficient for in vitro degradation by mu-calpain. J Biol Chem 1999;274(43):30874-81.
80. Okuda H, Hirai S, Takaki Y, Kamada M, Baba M, Sakai N, et al. Direct interaction of the beta-domain of VHL tumor suppressor protein with the regulatory domain of a typical PKC isotypes. Biochem Biophys Res Commun 1999;263(2):491-7.
81. Gottifredi V, Prives C. P53 and PML: new partners in tumor suppression. Trends Cell Biol 2001;11(5):184-7.
82. Ory D S, Neugeboren B A, Mulligan R C. A stable human-derived packaging cell line for production of high titer retrovirus/vesicular stomatitis virus G pseudotypes. Proc Natl Acad Sci USA 1996;93 (21):11400-6.
83. Iliopoulos O, Kibel A, Gray S, Kaelin W G, Jr. Tumour suppression by the human von Hippel-Lindau gene product. Nat Med 1995;1(8):822-6.
84. Wiltrout R H, Gregorio T A, Fenton R G, Longo D L, Ghosh P, Murphy W J, et al. Cellular and molecular studies in the treatment of murine renal cancer. Semin Oncol 1995;22(1):9-16.
85. Onda H, Lueck A, Marks P W, Warren H B, Kwiatkowski D J. Tsc2(±) mice develop tumors in multiple sites that express gelsolin and are influenced by genetic background. J Clin Invest 1999;104(6):687-95.
86. Kobayashi T, Minowa O, Kuno J, Mitani H, Hino O, Noda T. Renal carcinogenesis, hepatic hemangiomatosis, and embryonic lethality caused by a germ-line Tsc2 mutation in mice. Cancer Res 1999;59(6):1206-11.
87. Adams J M, Cory S. The Bcl-2 protein family: arbiters of cell survival. Science 1998;281 (5381):1322-6.
88. Zha J, Harada H, Yang E, Jockel J, Korsmeyer S J. Serine phosphorylation of death agonist BAD in response to survival factor results in binding to 14-3-3 not BCL-X(L). Cell 1996;87(4):619-28.
89. Bruick R K. Expression of the gene encoding the proapoptotic Nip3 protein is induced by hypoxia. Proc Natl Acad Sci USA 2000;97(16):9082-7.
90. Sowter H M, Ratcliffe P J, Watson P, Greenberg A H, Harris A L. HIF-1-dependent regulation of hypoxic induction of the cell death factors BNIP3 and NIX in human tumors. Cancer Res 2001;61(18):6669-73.
91. Lieberthal W, Triaca V, Koh J S, Pagano P J, Levine J S. Role of superoxide in apoptosis induced by growth factor withdrawal. Am J Physiol 1998;275(5 Pt 2):F691-702.
92. Knebelmann B, Ananth S, Cohen H T, Sukhatme V P. Transforming growth factor alpha is a target for the von Hippel-Lindau tumor suppressor. Cancer Res 1998;58(2):226-31.
93. de Paulsen N, Brychzy A, Fournier M C, Klausner R D, Gnarra J R, Pause A, et al. Role of transforming growth factor-alpha in von Hippel-Lindau (VHL)(−/−) clear cell renal carcinoma cell proliferation: a possible mechanism coupling VHL tumor suppressor inactivation and tumorigenesis. Proc Natl Acad Sci USA 2001;98(4):1387-92.
94. Wu G, Lee W H, Chen P L. NBS1 and TRF1 colocalize at promyelocytic leukemia bodies during late S/G2 phases in immortalized telomerase-negative cells. Implication of NBS1 in alternative lengthening of telomeres. J Biol Chem 2000;275(39):30618-22.
95. Bischof O, Kim S H, Irving J, Beresten S, Ellis N A, Campisi J. Regulation and localization of the Bloom syndrome protein in response to DNA damage. J Cell Biol 2001;153(2):367-80.
96. Ali R B, Teo A K, Oh H K, Chuang L S, Ayi T C, Li B F. Implication of localization of human DNA repair enzyme O6-methylguanine-DNA methyltransferase at active transcription sites in transcription-repair coupling of the mutagenic O6-methylguanine lesion. Mol Cell Biol 1998;18(3):1660-9.
97. Wang Z G, Ruggero D, Ronchetti S, Zhong S, Gaboli M, Rivi R, et al. PML is essential for multiple apoptotic pathways. Nat Genet 1998;20(3):266-72.
98. Sancar A. DNA repair in humans. Annu Rev Genet 1995;29:69-105.
99. Lee A S. The glucose-regulated proteins: stress induction and clinical applications. Trends Biochem Sci 2001;26(8):504-10.
100. McCullough K D, Martindale J L, Klotz L O, Aw T Y, Holbrook N J. Gadd153 sensitizes cells to endoplasmic reticulum stress by down-regulating Bcl2 and perturbing the cellular redox state. Mol Cell Biol 2001;21(4):1249-59.
101. Nakagawa T, Zhu H, Morishima N, Li E, Xu J, Yankner B A, et al. Caspase-12 mediates endoplasmic-reticulum-specific apoptosis and cytotoxicity by amyloid-beta. Nature 2000;403(6765):98-103.
102. Rao R V, Hermel E, Castro-Obregon S, del Rio G, Ellerby L M, Ellerby H M, et al. Coupling endoplasmic reticulum stress to the cell death program. Mechanism of caspase activation. J Biol Chem 2001;276(36):33869-74.
103. Lieberthal W, Levine J S. Mechanisms of apoptosis and its potential role in renal tubular epithelial cell injury. Am J Physiol 1996;271(3 Pt 2):F477-88.
104 Hammond C, Jeffers L, Carr B I, Simon D. Multiple genetic alterations, 4q28, a new suppressor region, and potential gender differences in human hepatocellular carcinoma. Hepatology 1999;29(5):1479-85.
   All references described herein are incorporated herein by reference in their entirety.

1. An isolated and purified DNA sequence comprising SEQ ID NO: 4 and antisense sequence of said DNA sequence.

2. An isolated and purified DNA sequence of at least 17 consecutive nucleotides of SEQ ID NO: 4 and antisense sequence of said DNA fragment.

3. An isolated and purified protein sequence comprising SEQ ID NO: 5 or a functional fragment thereof.

4. An isolated and purified protein fragment of at least 21 consecutive amino acids of SEQ ID NO: 5.

5. A diagnostic assay for detecting a disease in an individual in need thereof comprising the steps of:
a) obtaining a biological sample from an individual;
b) detecting expression of a wild type Jade family gene in the biological sample; and
c) comparing the expression of the wild type Jade family gene in the biological sample to expression of the wild type Jade family gene in a control sample wherein a change in level expression of the Jade family gene in the biological sample is indicative of a disease.

6. The assay of claim 5, wherein the detecting step b) is performed using an antibody against Jade family of proteins or an immunogenic portion thereof.

7. The assay of claim 5, where the change is a decrease in the level of Jade family of protein gene expression.

8. The assay of claim 5, wherein the change is an increase in the level of Jade family of protein gene expression.

9. The assay of claim 5 wherein the disease is cancer.

10. The assay of claim 9, wherein the cancer is a renal cancer.

11. The assay of claim 9, wherein the cancer is a colon cancer.

12. The assay of claim 9, wherein the cancer is a sarcoma.

13. The assay of claim 5 wherein the disease is cystic renal disease.

14. The assay of claim 13, wherein the cystic renal disease is autosomal dominant polycystic kidney disease type I (ADPKD-1).

15. The assay of claim 5, wherein the disease is a neurodegenerative disorder

16. The assay of claim 15, wherein the neurodegenerative disorder is Alzheimer's disease (AD).

17. The assay of claim 5, wherein the disease is an autoimmune disease.

18. The assay of claim 5, wherein the disease is heart disease.

19. An assay for screening for agents capable of modulating a Jade family of protein activity comprising the steps of:
a) providing a cell transfected with a vector comprising a nucleic acid sequence encoding Jade family of protein or a functional fragment thereof;
b) providing an apoptosis marker for detection of increased apoptosis;
c) contacting said cell with a test agent; and
d) comparing the amount of the apoptosis marker in the cell after step c) to the cell which is not contacted with a test agent wherein an increase in apoptosis is indicative of an agent which enhances Jade activity and a decrease in apoptosis is indicative of an agent which inhibits Jade activity.

20. An assay for screening for agents capable of modulating Jade family of protein activity comprising the steps of:
a) providing a cell expressing Jade protein or a fragment thereof;
b) contacting the cell with a test agent or a mixture thereof;
c) detecting the amount of Jade protein in the cell of step b) compared to a cell of step a) which is not contacted with the test agent, wherein difference in the amount of Jade in the cells of the steps a) and b) indicates an agent capable of modulating Jade activity.

21. The assay of claim 20, wherein the detecting in step c) is performed using an antibody against Jade protein or an immunogenic fragment thereof.

22. A vector comprising a nucleic acid encoding SEQ ID NO: 5.

23. A vector comprising a nucleic acid encoding SEQ ID NO: 1

24. A vector comprising a nucleic acid encoding SEQ ID NO: 2.

25. An antibody against a Jade family of protein or an antigenic epitope thereof.

26. An antibody against an amino acid sequence of the SEQ ID NO.:1.

27. An antibody against an amino acid sequence of the SEQ ID NO.:2.

28. An antibody against an amino acid sequence of the SEQ ID NO.:3.

29. A method of treating a disease or disorder with reduced Jade protein activity comprising administering a Jade activity increasing agent in a pharmaceutically acceptable carrier to an individual in need thereof.

30. The method of claim 29, wherein the Jade activity increasing molecule is a proteasome inhibitor.

31. The method of claim 30, wherein the proteasome inhibitor is other than MLN341.

32. The method of claim 29, 30, or 31, wherein the disease or disorder is selected from a group consisting of renal cancer, VHL disease, and renal cystic disease.

33. A method of treating a disease or disorder with an increased Jade protein activity comprising administering a Jade protein activity inhibitor in a pharmaceutically acceptable carrier to an individual in need thereof.

34. The method of claim 33, wherein the Jade protein activity inhibitor is selected from the group consisting of an antibody against the Jade protein or an immunogenic fragment thereof, an antisense nucleic acid directed against the nucleic acid encoding Jade protein or a fragment thereof, an interference RNA (iRNA) directed against the transcript encoding the Jade protein or a fragment thereof and an agent promoting destruction of Jade protein.

35. A method of evaluating clinical effect of a treatment in an individual receiving therapeutic proteasome inhibitors comprising the steps of:
a) obtaining a first biological sample from an individual before a first treatment with proteasome inhibitors;
b) obtaining a second biological sample from an individual after the first treatment; and
c) measuring Jade protein activity in the first and second biological samples, wherein increase in Jade protein activity in the second biological sample compared to the first biological sample indicates that the proteasome inhibitors used in the first treatment have a clinical effect.
